(19) United States
(12) Patent Application Publication
(10) Pub. No.: US 2011/0301447 A1
(43) Pub. Date: Dec. 8, 2011

(54) Versatile video interpretation, visualization, and management system

(75) Inventors: Sun Young Park, (US); Dustin Sargent, (US); Ulf Peter Gustafsson, (US); Wenjing Li, (US); Rolf Wolters, (US); Stephen D. Fleischer, (US)

(73) Assignee: STI Medical Systems, LLC

(21) Appl. No.: 13/134,507

(22) Filed: Jun. 7, 2011

Related U.S. Application Data
(60) Provisional application No. 61/397,169, filed on Jun. 7, 2010.

(51) Int. Cl. A61B 5/00; H04N 5/04

(52) U.S. Cl. 600/407; 348/500; 348/E05.009

(57) ABSTRACT

A process and device for detecting colon cancer by classifying and annotating clinical features in video data containing colonoscopic features by applying a probabilistic analysis to intra-frame and inter-frame relationships between colonoscopic features in spatially and temporally neighboring portions of video frames, and classifying and annotating as clinical features any of the colonoscopic features that satisfy the probabilistic analysis as clinical features. Preferably the probabilistic analysis is Hidden Markove Model analysis, and the process is carried out by a computer trained using semi supervised learning from labeled and unlabeled examples of clinical features in video containing colonoscopic features.

This application claims the benefit of U.S. provisional patent application No. 61/397,169 filed Jun. 7, 2010.

### TECHNICAL FIELD

The present invention generally relates to medical imaging, and more specifically to the interpretation, visualization, quality assessment, and management of endoscopy exams, videos, imaging and patient data.

### BACKGROUND ART

Although this invention is being disclosed in connection with video interpretation, quality assessment, visualization, and management in colonoscopy, it is applicable to other areas of medicine, including but not limited to, endoscopic procedures such as upper endoscopy, enteroscopy, bronchoscopy and endoscopic retrograde cholangiopancreatography.

According to the American Cancer Society's Cancer Facts and Figures (ACS, Cancer Facts and Figures, 2004, American Cancer Society, 2010, incorporated herein by reference), colorectal cancer is one of four cancers estimated to produce more than 100,000 new cancer cases per year. Colorectal cancer ranks second for new cancer cases in men and third for new cancer cases in women. Colorectal cancer is also the second leading cause of cancer-related death in the United States, causing more than 51,370 deaths annually. If colorectal cancer is not discovered before metastasis (or the spread of a disease from one organ or part to another non-adjacent organ or part), the five-year survival rate is less than 10% (L. Rabeneck, H. B, El-Serag, J. A. Davila, R. S. and Sandler, Outcomes of colorectal cancer in the United States: no change in survival (1986-1997), Am. J. Gastroenterol. 98(2), pp. 471-477, 2003, incorporated herein by reference). However, if colorectal cancer can be detected and treated while it is localized and in its early stage, the five year survival rate jumps to over 90%. Early diagnosis is of critical importance role for the patient's survival (S. Winawer, S., R. Fletcher, D. Rex, J. Bond, R. Burt, J. Ferrucci, T. Ganiats, T. Levin, S. Woolf, D. Johnson, L. Kirk, S. Litin, C. and Simmang, “Colorectal cancer screening and surveillance: clinical guidelines and rationale—update based on new evidence,” Gastroenterology 124(2), pp. 544-560. 2003; and S. Winawer, “The multidisciplinary management of gastrointestinal cancer. Colorectal cancer screening,” Best Pract. Res. Clin. Gastroenterol. 21(6), pp. 1031-1048, 2007, incorporated herein by reference).

The advantages of early detection of colorectal cancer clearly highlight the need for a colonoscopic video interpretation, visualization, and management system to enhance a physician's ability to detect colorectal disease. This system would preferably automatically interpret the colonoscopic video data and detect tissue anomalies such as polypoid lesions (polyps or an abnormal growth of tissue) and diverticulosis (outpocketings of the colonic mucosa and submucosa through weaknesses of muscle layers in the colon wall), provide information and feedback regarding the quality of the colonoscopic exam, and provide efficient capture, storage, indexing, search, and retrieval of a patient's colonoscopic exam and video data.

A fundamental function of such a system would be the application of computer algorithms to interpret the key features in the colonoscopic video data, referred to as “colonoscopic features.” A number of studies have investigated feature extraction, detection, classification, and annotation techniques to automate the diagnostic interpretation, segmentation (filtering into relevant sections), and presentation of colonoscopic features in images and videos. For example, Tjoa et al. (M. P. Tjoa and S. M. Krishnan, “Feature extraction for the analysis of colon status from the endoscopic images,” Biomed. Eng. Online 2:9, p. 38-42, 2003, incorporated herein by reference) extracted different statistical measurements from the texture spectra in the chromatic and achromatic domains, used principal component analysis to reduce the dimension of a feature vector, and evaluated the data using back-propagation neural networks. Karkanis et al. (S. A. Karkanis, D. K. Iakovidis, D. E. Maroulis, D. A. Karras, and M. Tzivras, “Computer-aided tumor detection in endoscopic video using color wavelet features,” IEEE Trans. Inf. Technol. Biomed. 7(3), pp. 141-152, 2003, incorporated herein by reference) applied a new feature called color wavelet covariance from wavelet decomposition to train and detect adenomatous polyps. Li et al. (P. Li, K. L. Chan, and S. M. Krishman, “Learning a multi-size patch-based hybrid kernal machine ensemble for abnormal region detection in colonoscopic images,” Proc. IEEE Computer Society Conference on Computer Vision and Pattern Recognition, 2, 2005, incorporated herein by reference) proposed to represent an image region using multi-size patches followed by constructing an ensemble based on a set of individual support vector classifiers to categorize the patches as normal or abnormal. Hwang et al. (S. Hwang, J. H. Oh, W. Tavanapong, J. Wong, and P. C. de Groen, “Polyp detection in colonoscopy video using elliptical shape feature,” Proc. International Conference on Image Processing (ICIP2007), pp. 465-468, 2007, and S. Hwang, J. H. Oh, W. Tavanapong, J. Wong, and P. C. de Groen, “Polyp detection in colonoscopy video using elliptical shape feature,” Proc. IEEE International Conference on Image Processing, 2, p. 468, 2007, incorporated herein by reference) utilized shape-based methods and fitted elliptical shapes into segmented regions by utilizing watershed based image segmentation and ellipse fitting algorithms. Dhandra et al. (B. V. Dhandra, R. Hegadi, M. Hangarge, and V. S. Malemath, “Analysis of abnormality in endoscopic imges using combined hsi color space and watershed segmentation”, in Proc. 18th International Conference on Pattern Recognition, pp. 695-698, 2006, incorporated herein by reference) applied morphological watershed segmentation in which the output indicated whether an endoscopic image was normal or abnormal based on the number of watershed regions. Zhao et al. (L. Zhao, C. P. Botha, J. O. Bescos, R. Truyen, F. M. Vos, and F. H. Post, “Lines of curvature for polyp detection in virtual colonoscopy,” IEEE Transactions on Visualization and Computer Graphics 12(5), pp. 885-832, 2006, incorporated herein by reference) introduced a novel polyp detection approach by employing a three-dimensional volume model and characterizing polyps using lines of curvature. Vilarifio et al. (F. Vilariño, G. Lacey, J. Zhou, H. Mulcahy, and S. Patchett, “Automatic labeling of colonoscopy video for cancer detection,” Lecture Notes in Computer Science 4477, pp. 290-297, 2007, incorporated herein by reference) proposed an image labeling algorithm using support vector machines and self organizing maps to detect cancerous polyps in colonoscopy video. In the Vilaririo study, the movements of a physician's eyes were tracked, and it was hypothesized that the physician's gaze would be drawn to salient image features and that sustained fixations would be associated with disease features. Cao et al. (Y. Cao, D. Li, D., W. Tavanapong, J. H. Oh, J. Wong, and P. C. de Groen, “Parsing and browsing tools for colonoscopy videos,” Proc. 12th annual ACM international conference on Multimedia, pp. 844-851, 2004, incorporated herein by reference) introduced spatio-temporal analysis techniques to automatically identify video segments (relevant sections) corresponding to diagnostic or therapeutic operations.

According to published results, the above-mentioned methods achieve good classification results. However, the generality of these results on all types of colonoscopic video data are questionable because the sample sets used for testing and training are relatively small, typically ranging from a few to about 100 video frames. Most of the above-mentioned methods are also trained using a set of pre-selected still images. A reliable extraction, detection, and classification system should, on the other hand, be based on a large set of images containing different types of abnormalities, as well as various obstructions, such as blood, stool, water, and therapeutic tools.

Furthermore, previous methods and approaches have not made use of intra-frame and inter-frame relationships between different features present in the colonoscopic video data that play an essential role in the present invention. In addition, no colonoscopy video interpretation system has previously considered two crucial aspects for clinical applications: data variations between patients, operations and devices, and multi-modality colonoscopy video data.

Methods and approaches to filter, index, parse and browse colonoscopic video data into different segments (relevant sections) based on content have been previously presented (S. Hwang, J. Oh, J. Lee, W. Tavanapong, P. C. de Groen, and J. Wong, “Informative frame classification for endoscopy video,” Medical Image Analysis 11(2), pp. 110-127, 2007; Y.-H. An, S. Hwang, J. Oh, W. Tavanapong, P. C. de Groen, J. Wong, and J. K. Lee. “Informative frame filtering in endoscopy videos,” Proc. SPIE 5747, pp. 291-302, 2005; and Y. Cao, D. Li, W. Tavanapong, J. Wong, J. Oh, and P. C. de Groen, “Parsing and browsing tools for colonoscopy videos,” Proc. 12th Annual ACM International Conference on Multimedia, pp. 844-851, 2004, incorporated herein by reference). There are also commercially available endoscopic video systems, which have viewing, recording, storage and retrieval capabilities (such as the Image Stream Medical nStream+ HD image management system, KayPentax Digital Video Recording System, Sony BZM D-1000 ImageCore HD Digital Capture System, Storz AIDA Compact II System, Storz AIDA with DICOM and HL7 Interface, Stryker Digital Capture HD and Ultradevices, and Summit Imaging EndoManager and EndoGI, incorporated herein by reference).

Research, capturing, analysis and annotation tools have also been developed, for example, the Arthemis software (D. Liu, Y. Cao, K. H. Kim, S. Stanek, B. Doungratanaex-Chai, K. Lin, K. W. Tavanapong, J. Wong, and P. C. de Groen, “Arthemis: Annotation software in an integrated capturing and analysis system for colonoscopy,” Comput. Methods Programs Biomed., 88(2), pp. 152-163, 2007, incorporated herein by reference). This software provides several functions for accessing video data, such as pausing and jumping to a specific video data frame, and pre-viewing video data at a fast rate. It can extract potentially important video data segments (relevant sections) using verbal dictation from the endoscopist that is recorded during the colonoscopy, and facilitates annotation according to the minimal standard terminology for endoscopy (L. Aabacken, B. Rembacken, O. LeMoine, K. Kuznetsov, J.-F. Rey, T. Rosch, G. Eisen, P. Cotton, and M. Fujino, “Minimal standard terminology for gastrointestinal endoscopy—MST 3.0,” Organization Mondiale Endoscopia Digestive, Committee for Standardization and Terminology, 2008, incorporated herein by reference), which offers a standardized selection of terms and attributes for the description of findings, procedures and complications.

However, these methods, systems and approaches provide only primitive video accessing functions already included in many generic video software packages. These systems also rely on manual dictation from an endoscopist, a time-consuming and expensive process. Furthermore, only rudimentary indexing, search and retrieval functions are provided, which limit their usefulness in the interpretation, visualization and management of both pre-recorded and new colonoscopic video data. Therefore, the clinically valuable information contained in colonoscopic video data is not being extracted and used to the fullest extent possible to improve patient care.

The following patents and patent applications may be considered relevant to the field of the invention:

U.S. Pat. No. 5,797,396 to Geiser et al., incorporated herein by reference, discloses an automated method for quantitatively analyzing digital images of approximately elliptical body organs, and in particular, two-dimensional echocardiographic images.

U.S. Pat. No. 5,999,840 to Grimson et al., incorporated herein by reference, discloses an image data registration method and system for the registering of three-dimensional surgical image data utilized in image guided surgery and frameless stereotaxy.

U.S. Pat. No. 6,106,470 to Geiser et al., incorporated herein by reference, discloses a method and apparatus for calculating the distance between ultrasound images using the sum of absolute differences.

U.S. Pat. No. 6,167,295 to Cosman, incorporated herein by reference, discloses an apparatus involving optical cameras and computer graphic means for the registering of anatomical subjects seen in the cameras, to compute graphic image displays of image data taken from computer tomography, magnetic resonance imaging or other scanning image means.

U.S. Pat. No. 6,456,735 to Sato et al., incorporated herein by reference, discloses an image display method and apparatus which enables the observation of a wide range of the wall surface of a three-dimensional tissue in one screen.

U.S. Pat. No. 6,484,049 to Seeley et al., incorporated herein by reference, discloses a fluoroscopic tracking and visualization system as an aid in intraoperative or perioperative imaging, in which images are formed of a region of the patient's body and a surgical tool or instrument is applied, and wherein the images aid in an ongoing procedure.

U.S. Pat. No. 6,490,475 to Seeley et al., incorporated herein by reference, discloses a fluoroscopic tracking and visualization system as an aid in intraoperative or perioperative imaging, in which images are formed of a region of the patient's body and a surgical tool or instrument is applied, and wherein the images aid in an ongoing procedure.

U.S. Pat. No. 6,514,207 to Ebadollahi et al., incorporated herein by reference, discloses methods and a system for processing an echocardiogram video of a patient's heart.

U.S. Pat. No. 6,975,755 to Baumberg, incorporated herein by reference, discloses an image processing method and apparatus for the detection and matching of features in images and identifying features in images for the purpose of indexing or categorization.

U.S. Pat. No. 6,735,465 to Panescu, incorporated herein by reference, discloses a process of refining a map of a body cavity as an aid in guiding and locating diagnostic or therapeutic elements on medical instruments positioned in a body.

U.S. Pat. No. 6,856,826 to Seeley et al., incorporated herein by reference, discloses a fluoroscopic tracking and visualization system for surgical imaging and displdy.

U.S. Pat. No. 6,856,827 to Seeley et al., incorporated herein by reference, discloses a fluoroscopic tracking and visualization system for surgical imaging and display of tissue structures of a patient.

U.S. Pat. No. 6,885,702 to Goudezeune et al., incorporated herein by reference, discloses a method for synchronization of the spatial position of a video image, in order to recover the position of an initial grid for initial digital coding of the said image by coding blocks, as well as a method of at least partial identification of the time-based syntax of the initial coding.

U.S. Pat. No. 6,895,267 to Panescu et al., incorporated herein by reference, discloses systems and methods for guiding and locating functional elements on medical devices positioned in a body as part of invasive diagnostic or therapeutic procedures.

U.S. Pat. No. 7,011,625 to Shar, incorporated herein by reference, discloses a method and system for accurately visualizing and measuring endoscopic images, by mapping a three-dimensional structure to a two-dimensional area using a plurality of endoscopic images of the structure.

U.S. Pat. No. 7,035,435 to Li et al., incorporated herein by reference, discloses a method and system for automatically summarizing a video document by decomposing the document into scenes, shots and frames, assigning an importance value to each scene, shot and frame, and allocating key frames based on the importance value of each shot in response to user input.

U.S. Pat. No. 7,047,157 to Li, incorporated herein by reference, discloses methods of processing and summarizing video content, including detection of key frames in the video, detection of events that are important for the particular video content, and manual segmentation of the video.

U.S. Pat. No. 7,162,292 to Ohno et al., incorporated herein by reference, discloses a beam scanning probe for surgery which can locate a site of a tumor to be treated in an effort to ease the surgery.

U.S. Pat. No. 7,203,635 to Oliver et al., incorporated herein by reference, discloses a system and methodology providing layered probabilistic representations for sensing, learning, and inference from multiple sensory streams at multiple levels of temporal granularity and abstraction. Based on an architecture of layered hidden Markov models (LHMMs), the invention facilitates robustness to subtle changes in environment and enables model adaptation with minimal retraining.

U.S. Pat. No. 7,209,536 to Walter et al., incorporated herein by reference, discloses a method and system of computed tomography colonography that includes the acquisition of energy sensitive or energy-discriminating computed tomography data from a colorectal region of a subject. Computed tomography data is acquired and decomposed into basis material density maps and used to differentiate and enhance contrast between tissues in the colorectal region. The invention is particularly applicable with the detection of colon polyps without cathartic preparation or insufflation of the colorectal region. The invention is further directed to the automatic detection of colon polyps.

U.S. Pat. No. 7,231,135 to Esenyan et al., incorporated herein by reference, discloses a computer-based video recording and management system used in conjunction with medical diagnostic equipment. The system allows a physician or medical personnel to record and time-mark significant events during a medical procedure on video footage, to index patient data with the video footage, and then to later edit or access the video footage with patient data from a database in an efficient manner. The system includes at least one input device that inserts a time-mark into the video footage, and a workstation that associates an index with each time-mark, extracts a portion of the video footage at the time-mark beginning just before and ending just after the time-mark, concatenates the portion of the video footage with other portions of video footage, into a shortened summary video clip, and stores both the video footage and summary video clip into a searchable database.

U.S. Pat. No. 7,263,660 to Zhang et al., incorporated herein by reference, discloses a system and method for producing a video skim by identifying one or more key frames from a video shot.

U.S. Pat. No. 7,268,917 to Watanabe et al., incorporated herein by reference, discloses image correction processing apparatus for correcting a pixel value of each pixel constituting image data obtained from an original image affected by peripheral light-off.

U.S. Pat. No. 7,355,639 to Lee, incorporated herein by reference, discloses a lens correction method for use on the processed output of an image sensor.

U.S. Pat. No. 7,382,244 to Donovan et al., incorporated herein by reference, discloses a video surveillance, storage, and alerting system utilizing surveillance cameras, video analytics devices, audio sensory devices, other sensory devices, and data storage devices.

U.S. Pat. No. 7,489,342 to Xin et al., incorporated herein by reference, discloses a system and method of managing multi-view videos by indexing temporal reference pictures, spatial reference pictures and synthesized reference pictures of the multi-view videos, and predicting each current frame of the multi-view videos based on the reference pictures.

U.S. Pat. No. 7,545,954 to Chan et al., incorporated herein by reference, discloses an event recognition system as part of a video recognition system. The system includes a sequence of continuous vectors and a sequence of binarized vectors. The sequence of continuous vectors represents spatial-dynamic relationships of objects in a pre-determined recognition area. The sequence of binarized vectors is derived from the sequence of continuous vectors by utilizing thresholds for determining binary values for each spatial-dynamic relationship. The sequence of binarized vectors indicates whether an event has occurred.

U.S. Pat. No. 7,561,733 to Vilsmeier et al., incorporated herein by reference, discloses a method and device for patient registration with video image assistance, wherein a spatial position of a patient and a stored patient data set are reciprocally assigned.

U.S. Pat. No. 7,570,791 to Frank et al., incorporated herein by reference, discloses a method and apparatus for performing two-dimensional to three-dimensional registration of image data used during image guided surgery by utilizing an initialization step and a refinement step.

U.S. Pat. No. 7,630,529 to Zalis, incorporated herein by reference, discloses a virtual colonoscopy system which includes a system for generating digital images, a storage device for storing the digital images, a digital bowel subtraction processor coupled to the storage device to receive images of a colon and for removing the contents of the colon from the image, and an automated polyp detection processor coupled to receive images of a colon from the storage device and for detecting polyps in the colon image.

U.S. Pat. Nos. 6,497,784 and 7,613,365 to Wang et al., incorporated herein by reference, discloses a video summarization system and method by computing the similarity between video frames to obtain multiple similarity values, extracting key sentences from the video frames, mapping the sentences into sentence vectors, computing the distance between each sentence vector to obtain distance values, dividing the sentences into clusters according to the distance values and the importance of the sentences, splitting the cluster with the highest importance into multiple new clusters, and extracting multiple key sentences from the clusters.

U.S. Pat. No. 7,627,823 to Takahashi et al., incorporated herein by reference, discloses a video information editing method and device which splits a video title into shots or scenes with time codes, performs semantic evaluation of the story, and adds the information from the evaluation to the respective scenes to organize a scene score.

U.S. Pat. No. 7,639,896 to Sun et al., incorporated herein by reference, discloses a multi-modal image registration method using compound mutual information.

U.S. Pat. No. 7,671,894 to Yea et al., incorporated herein by reference, discloses a method and system for processing multi-view videos for view synthesis using skip and direct modes.

European Patent No. EP 2054852 B1 to Jia Gu et al., incorporated herein by reference, discloses image processing and computer aided diagnosis for diseases, such as colorectal cancer, using an automated image processing system providing a rapid, inexpensive analysis of video from a standard endoscope, and a three-dimensional reconstructed view of the organ of interest, such as a patient's colon.

U.S. Patent Application Publication No. 2002/0181739 to Hallowell et al., incorporated herein by reference, discloses a video system for monitoring and reporting weather conditions by receiving a sequential series of images, maintaining and updating a composite image which represents a long-term average of the monitored field of view, applying edge-detection filtering on the received and composite images, extracting persistent edges existing in both the received and composite image, and using this edge information to predict a weather condition.

U.S. Patent Application Publication No. 2006/0004275 to Vija et al., incorporated herein by reference, discloses systems and methods for co-registering, displaying and quantifying images from numerous different medical modalities utilizing multiple user-defined regions-of-interest.

U.S. Patent Application Publication No. 2006/0293558 to De Groen et al., incorporated herein by reference, discloses a computer-based method that allows automated measurement of a number of metrics that likely reflect the quality of a colonoscopic procedure. The method is based on analysis of a digitized video file created during colonoscopy, and produces information regarding insertion time, withdrawal time, images at the time of maximal intubation, the time and ratio of clear versus blurred or non-informative images, and a first estimate of effort performed by the endoscopist.

U.S. Patent Application Publication No. 2007/0081712 to Huang et al., incorporated herein by reference, discloses a learning-based framework for whole body landmark detection, segmentation, and change detection is single-mode and multi-mode medical images.

U.S. Patent Application Publication No. 2007/0232868 to Reiner, incorporated herein by reference, discloses a quality assurance system and method that generates a quality assurance scorecard for radiologists that use digital devices in radiological-based medical imaging.

U.S. Patent Application Publication No. 2007/0171220 and 2007/0236494 to Kriveshko, incorporated herein by reference, discloses an improved scanning system by acquiring three-dimensional images as an incremental series of fitted three-dimensional data sets, testing for successful incremental fits in real time, and providing a variety of visual user cues and process modifications depending upon the relationship of newly acquired data to previously acquired data.

U.S. Patent Application Publication No. 2007/0258642 to Thota, incorporated herein by reference, discloses a unique system, method, and user interface that facilitates more efficient indexing and retrieval of images by utilizing a geo-code annotation component that annotates at least one image with geographic location metadata; and a map-based display component that displays one or more geo-coded images on a map according to their respective locations.

U.S. Patent Application Publication No. 2008/0009674 to Yaron, incorporated herein by reference, discloses a method and system for navigating within a flexible organ of the body of a patient by employing a global three-dimensional (3D) model of the flexible organ.

U.S. Patent Application Publication No. 2008/0030578 to Razzaque et al., incorporated herein by reference, discloses a system and method of providing composite real-time dynamic imagery of a medical procedure site from multiple modalities, which continuously and immediately depicts the current state and condition of the medical procedure site synchronously with respect to each modality and without undue latency.

U.S. Patent Application Publication No. 2008/0058593 to Jia Gu et al., incorporated herein by reference, discloses a process for providing computer aided diagnosis from video data of an organ during an examination with an endoscope, by analyzing and enhancing image frames from the video, creating three dimensional reconstruction of the organ and detecting and diagnosing any lesions in the image frames in real time during the examination.

U.S. Patent Application Publication No. 2008/0118135 to Averbush et al., incorporated herein by reference, discloses an adaptive navigation technique for navigating a catheter through a body channel or cavity using an assembled three-dimensional image.

U.S. Patent Application Publication No. 2008/0175486 to Yamamoto, incorporated herein by reference, discloses a video-attribute-information output apparatus, video digest forming apparatus, computer program product, and video-attribute-information output method.

U.S. Patent Application Publication No. 2009/0028403 to Bar-Aviv et al., incorporated herein by reference, discloses a system for analyzing a source medical image of a body organ that includes an input unit for obtaining the source medical image having three dimensions or more, a feature extraction unit that is designed for obtaining a number of features of the body organ from the source medical image, and a classification unit that is designed for estimating a priority level according to the features.

U.S. Patent Application Publication No. 2009/0136141 to Badawy et al., incorporated herein by reference, discloses a quick and efficient method for analyzing a segment of video data by acquiring a reference portion from a reference frame, acquiring subsequent portions from a corresponding subsequent reference frame, comparing the subsequent portion with the reference portion and detecting an even based upon the comparison.

U.S. Patent Application Publication No. 2009/0220133 to Sawa et al., incorporated herein by reference, discloses a medical image processing apparatus and method for the detection of locally protruding lesions.

U.S. Patent Application Publication No. 2009/0279759 to Sirohey et al., incorporated herein by reference, discloses a system and method for synchronizing corresponding locations among multiple images of an object to identify and suppress particles in virtual dissection data for an anatomical structure.

U.S. Patent Application Publication No. 2009/0304248 to Zalis et al., incorporated herein by reference, discloses a structure-analysis system, method, software arrangement and computer-accessible medium for digital cleansing of computed tomography colonography images.

U.S. Patent Application Publication No. 2009/0315978 to Wurmlin et al., incorporated herein by reference, discloses a method for generating a three-dimensional representation of a dynamically changing three-dimensional scene by acquiring synchronized video streams, determining camera parameters, tracking the movement of objects, determining the identity of the objects in the video streams, and determining the three-dimensional position of the objects by combining the information from the video streams.

International Patent Application No. WO 2007/048091 to Zalis et al., incorporated herein by reference, discloses a system, method, software arrangement and computer-accessible medium for performing electronic cleansing of computer tomography colonography images.

International Patent Application No. WO 2007/084589 to Kriveshko et al., incorporated herein by reference, discloses an improved scanning system by acquiring three-dimensional images as an incremental series of fitted three-dimensional data sets, testing for successful incremental fits in real time, and providing a variety of visual user cues and process modifications depending upon the relationship of newly acquired data to previously acquired data.

Japanese Patent Application No. JP 2009109508 to Morimoto et al., incorporated herein by reference, discloses a system and device to detect a person in a sensing area without any erroneous detection.

Accordingly, it is an object of the present invention to provide a process and device for automatically detecting key features in video, such as clinical features in colon video frames containing colonoscopic features, and classifying and annotating (specifying location) the clinical features in the video frame.

It is a further object of the present invention to provide such a process and device that can be trained economically on a large sample set of data to improve reliability.

It is a still further object of the present invention to provide unsupervised detection and tracking of clinical features, such as colonic polyps and diverticula, in colonoscopic videos.

It is a still further object of the present invention to provide the ability to longitudinal exam review of two colonoscopic videos.

### DISCLOSURE OF INVENTION

The above and present objects are achieved by obtaining multiple colonoscopy video frames containing colonoscopic features and applying a probabilistic analysis to intra-frame relationships between colonoscopic features in spatially neighboring portions of the video frames, and to inter-frame relationships between colonoscopic features in temporally neighboring portions of the video frames, and then classifying and annotating as clinical features any of the colonoscopic features that satisfy the probabilistic analysis as clinical features. The probabilistic analysis is preferably selected from the group consisting of Hidden Markov Model analysis and a conditional random field classifier. Preferably also, the process comprises training a computer to perform the probabilistic analysis by semi supervised learning from labeled and unlabeled (including, without limitation, annotated and unannotated) examples of clinical features in video frames containing colonoscopic features. Preferably also, the training comprises physician feedback.

The process further comprises applying a forward-backward algorithm and model parameter estimation. Preferably, the process is augmented by additionally applying augmenting probabilistic analysis to at least one additional dimension of relationships between the colonoscopic features selected from the group consisting of frame quality, anatomical structures, and imaging multimodality. Preferably, the additional applying step is applied in a hierarchical manner first to video quality, then to anatomical structures, then to multimodalities.

In a preferred embodiment, the process comprises training a computer to perform probabilistic analysis by semi supervised learning from labeled and unlabeled examples of clinical features in video frames containing colonoscopic features, obtaining multiple colonoscopy video frames containing colonoscopic features, excluding any uninformative video frames, applying a probabilistic analysis selected from the group consisting of Hidden Markov Model analysis and conditional random field classifier to five dimensions of relationships between colonoscopic features in temporally or spatially neighboring portions of the video frames. The five dimensions of relationships consist of inter-frame relationships, intra-frame relationships, frame quality, anatomical structures, and imaging modalities. Finally, the process comprises classifying and annotating any of the colonoscopic features in the video frames that satisfy the probabilistic analysis as clinical features.

Preferably, the process further comprises pre-processing the video frames before the applying step, wherein the pre-processing step is selected from the group consisting of detecting glare regions, detecting edges, detecting potential tissue boundaries, correcting for optical distortion, de-interlacing, noise reduction, contrast enhancement, super resolution and video stabilization.

Preferably, the process further comprises providing progressively decreasing weighting scores as the field of view of the video frames increases.

The process preferably further comprises filtering the video frames into clinically relevant and clinically irrelevant sections and displaying or storing only frames that exceed a threshold for clinical relevance, wherein the filtering is performed by analyzing the video frames to estimate at least one measure of content of each video frame; aggregating frames into sections of similar content measure; and performing at least one action on frames that exceed a threshold for the clinical relevance metric, wherein clinical relevance of the content of each frame is scored according to a metric for that action.

The process further comprises providing a generic digital colon model for visual navigation through colon videos, and preferably clinical features are registered within the generic digital colon model.

The invention further comprises tracking annotated clinical features in subsequent video frames.

The invention further comprises a process for video spatial synchronization of colonoscopic videos, including tagging spatially and temporally coarsely spaced video frames with spatial location information in each video; estimating positions of frames subsequent to the tagged video frames in each video; and registering frames in the videos having most closely matching features.

The device of the invention comprises obtaining means for obtaining multiple colonoscopy video frames containing colonoscopic features; excluding means for excluding any uninformative video frames; applying means for applying a probabilistic analysis selected from the group consisting of Hidden Markov Model analysis and conditional random field classifier to five dimensions of relationships between colonoscopic features in temporally or spatially neighboring portions of said video frames; wherein the five dimensions of relationships consist of inter-frame relationships, intra-frame relationships, frame quality, anatomical structures, and imaging multimodalities; classifying and annotating means for classifying and annotating any of the colonoscopic features in the video frames that satisfy the probabilistic analysis as clinical features; filtering means for creating sections of said video containing relevant clinical features. Preferably, the probabilistic analysis has been trained by semi supervised learning from labeled and unlabeled examples of clinical features in video containing colonoscopic features, and the device further includes storage means for capturing, storing, searching and retrieving clinically relevant video frames; feature alert means for automatically interpreting, classifying and annotating the video frames; and field of view scoring means for scoring field of view of the video frames.

### BRIEF DESCRIPTION OF DRAWINGS

FIG. 1 depicts a schematic of the video interpretation system of the current invention.

FIG. 2 displays relationships in terms of strong (S), average (A), and weak (W) between the colonoscopic features of blur (40), glare (41), illumination (42), blood (50), stool (51), surgical tools (52), water (53), diverticula (60), mucosa (61), lumen (62), and polyps (63).

FIG. 3 is a graphical representation of a two level Hidden Markov Model (HMM) connecting the intra-frame and inter-frame relationships.

FIG. 4 illustrates the probabilistic relationships between state transitions and observations of the second-level HMM with T1, T2 and T3 depicting three state transitions, O1, O2, and O3 depicting the observations of colonoscopic features in the video data, and p1, p2, and p3 being the conditional probabilities of observing the clinical features in a training dataset.

FIG. 5 illustrates the structure and the probabilistic state transition of the data quality EHMM with I10, I11, and I12 depicting different informative states, U30 and U31 depicting uninformative states, and p and q being the state transition probabilities from ‘informative to uninformative’ and ‘uninformative to informative’, respectively.

FIG. 6 illustrates the anatomical colon segments (rectum (10), sigmoid colon (11), descending colon (12), transverse colon (13), ascending colon (14), and cecum (15)) and colon landmarks (anus (20), sigmoid/descending colon transition (21), splenic flexure (22), hepatic flexure (23), ileocecal valve (24), and appendiceal orifice (25)) utilized by the anatomical EHMM.

FIGS. 7(a) and 7(b) generally illustrates a digital colon model with a colonoscopic video view.

FIG. 7(a) displays the generic colon and the location of the tip (100) of the colonoscope during a colonoscopy.

FIG. 7(b) shows the colonoscopic video view at the location of the tip (100) of the colonoscope (see FIG. 7(a)) during a colonoscopy.

FIG. 8(a)-(f) generally illustrates the incorporation of microscopic and spectroscopic probe data into the digital colon model.

FIG. 8(a) shows the digital colon model with the position of the colonoscope tip (100) and the probe locations (200) where the probe is (or was used).

FIG. 8(b) shows the traditional colonoscopic video view with the probe tip (300) extended into the video view.

FIG. 8(c) depicts the location of the microscopic (310) probe data superimposed onto the digital colon model.

FIG. 8(d) displays the magnified view of the microscopic imaging data (310) such as acquired from confocal microscopy or optical coherence tomography.

FIG. 8(e) depicts the location of the spectroscopic (320) probe data superimposed onto the digital colon model.

FIG. 8(f), displays the spectroscopic data (320) such as acquired from infrared spectroscopy.

FIG. 9(a)-(d) generally display the output of the feature alert system.

FIG. 9(a) displays no detection.

FIG. 9(b) displays the initial detection as a black box of fine lines around the feature.

FIG. 9(c) displays a higher probability of detection with a black box of medium lines around the feature.

FIG. 9(d) displays the highest probability of detection with a black box of coarse lines around the feature.

FIG. 10 shows the algorithm flowchart for detection and tracking of polyps (abnormal growth of tissue) or diverticula (outpuching of a hollow structure) in colonoscopic videos.

FIG. 11(a)-(d) generally display the output of the polyp and diverticula detection and tracking system.

FIG. 11(a) displays no detection.

FIG. 11(b) displays detection as an ellipse of fine lines around the feature.

FIG. 11(c) displays first tracking with an ellipse of medium lines around the feature.

FIG. 11(d) displays continued tracking with an ellipse of coarse lines around the feature.

FIG. 12 displays the flowchart for video filtering of colonoscopic video.

FIG. 13 graphically depicts one possible embodiment of the video aggregation step of colonoscopic video filtering.

FIG. 14 graphically depicts one possible embodiment of the action execution step of colonoscopic video filtering.

FIG. 15 displays the flowchart for video synchronization of two colonoscopic videos (video A and B).

FIG. 16(a)-(b) generally displays the scoring of the field of view visualization scoring system.

FIG. 16(a) graphically depicts one possible embodiment of the field of view visualization scoring system for a single colonoscopic video frame. The 60° center field of view is assigned a score of 1.0 and each twenty degree increase in field of view decreases the score by 0.25.

FIG. 16(b) graphically depicts one possible embodiment of the field of view visualization scoring system for sections of as well as for an entire colonoscopic exam. Different sections of the colon are assigned scores (0.6, 0.8, 1.0, 0.5, 0.8, 0.9, 0.9, 0.7, and 0.9 based on the scores for the single frames (see FIG. 16(a)). The exam score is the average of the score for the different video sections (0.78).

### BEST MODES FOR CARRYING OUT INVENTION

The presently preferred embodiment of the invention discloses an interpretation, visualization, and management system for colonoscopic patient exam and video data.

The video interpretation system preferably identifies and annotates (specifies location within a frame) key colonoscopic features in frames of colonoscopic video data by applying an innovative multi-layer Semi-Supervised Embedded Hidden Markov Model (SSEHMM). The SSEHMM models the spatial and temporal relationships between colon findings, data quality, anatomical structures and imaging modalities within and between video data frames. The SSEHMM is preferably trained using semi-supervised learning. In computer science, semi-supervised learning is a class of machine learning techniques that make use of both labeled and unlabeled data for training—typically a small amount of labeled data with a large amount of unlabeled data. In the present invention, the semi-supervised learning increases the amount of available training data by using unlabeled videos. The system collects feedback from physicians about the relevance of the output to ensure that the system annotations match physician interpretation. This allows the model to effectively account for variations between patients and procedures when there is only a limited amount of training data available.

The video visualization and management system preferably provides capture, storage, search, and retrieval functionality of all patient, exam, and video information. The system also preferably applies image enhancement technologies to improve visualization of abnormal findings in the colon, and preferably includes a generic digital colon model that enables visual navigation through colon videos. A feature alert system that automatically interprets the colon video and classifies and annotates the findings, and a screening system that detects and tracks the diagnostically important features of polyps and diverticula, are also preferably included. Other important components include a segmentation (sometimes referred to as “filtering”, to avoid ambiguity) method that filters colon exam video data into clinically relevant or irrelevant segments (relevant sections), and a method for synchronizing (registering) exam video data to the generic colon model for longitudinal exam comparisons. Finally, the system also preferably includes a field of view scoring system that assesses the adequacy of the exam.

### Video Interpretation System

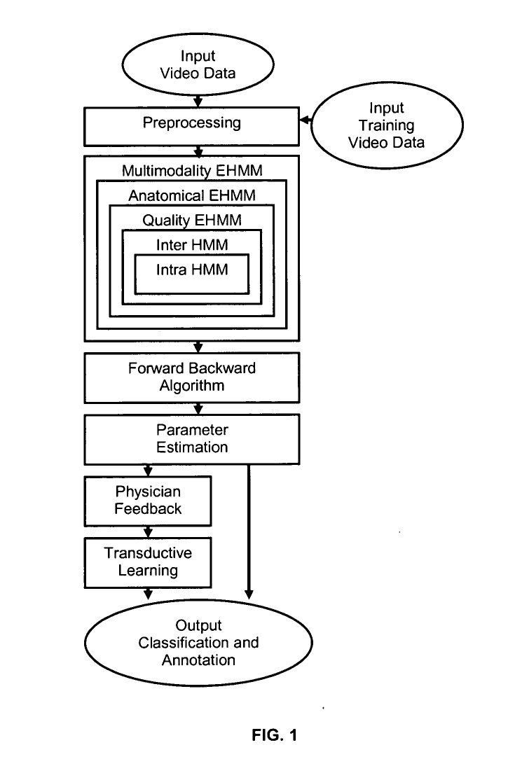

A schematic of the preferred embodiment of the video interpretation system of the present invention is illustrated in FIG. 1 The core component of this system is a SSEHMM (Semi-Supervised Embedded Hidden Markov Model) which preferably combines a novel hierarchical extension of the HMM (Hidden Markov Model) and an application of semi-supervised learning to time-sequence data. Although the preferred embodiment utilizes the HMM, any other probabilistic analysis methods with the Markov property can be used.

Five different relationships between colonoscopic features can be identified in colon videos, each of which is effectively and efficiently incorporated into the SSEHMM:
1. The spatial relationships between colonoscopic features in a single video frame (Intra Frame).
2. The time-course or temporal relationship between colonoscopic features in neighboring video frames (Inter Frame).
3. The relationship between video frames of different quality (Frame Quality).
4. The relationship between video frames from different anatomical segments in the colon (Anatomical Structure).
5. The relationship between different imaging modalities such as white-light reflectance, narrow band reflectance, fluorescence, and chromo-endoscopy (Imaging Multimodality).

The Markov property of the model inherently incorporates neighborhood information in both space and time (Intra Frame and Inter Frame), and the embedding scheme uses Frame Quality, Anatomical Structure and Imaging Modality, all to model the multi-dimensionality of the above five relationships in an explicit and computationally efficient manner.

In probability theory, the Markov property states that the probability distribution of future states of a random process (such as a stream of video images) depends only on the current state but not on the previous state or states. In a regular Markov model the current and future states of the random process are directly visible and, thus, can be observed in the video scene. The parameters in a regular Markov model are thus the transition probabilities between the current and future states. Conversely, in a HMM, the states are not directly observable (they are hidden); instead there is a set of observations about the current and future states that are probabilistically related. Thus, the state sequence is hidden and can only be inferred through the observations. The parameters of a HMM are therefore the probabilities relating the observations to the states, and the transition probabilities between the states.

The hierarchical HMM design is based on an important observation about colonoscopy video, namely that there is a higher probability to detect features when the features have been detected in adjacent frames.

Furthermore, differences in video data quality and video data properties, according to colon anatomy, indicate that different HM Ms should be applied to different colon segments. The present invention takes this into account by embedding HMMs (EHMMs) in other HMMs. An embedded HMM is a generalized HMM with a set of so called superstates, each of which is itself an HMM. The present invention also models the relationship between different imaging modalities, such as regular white light, narrow band, fluorescence, and chromo endoscopy, so that the applicability of the EHMM is further increased. To the best of the inventor's knowledge and belief, this is the first report that incorporates all five of these relationships into a video interpretation system by utilizing EHMM.

Inter-patient variations significantly degrade the generalization capability of inductive learning techniques in medical applications. Inductive learning techniques learn classification functions from training data. Therefore, inductive classifiers have poor predictive accuracy when trained with data which does not adequately represent the entire population. Medical video data and colonoscopy video data in particular, suffer from this problem; although there is a large amount of video available, annotated training video is comparatively rare and expensive to produce. To address this drawback, the EHMM is trained by semi-supervised learning. Semi-supervised learning is an alternate learning method in which both labeled and unlabeled examples can be used for training. This vastly increases the size of the training set, allowing the training data to better represent the underlying population.

### Features

The video interpretation system preferably classifies and annotates colonoscopic video frames and segments (relevant sections) according to the minimal standard terminology for endoscopy (L. Aabacken, B. Rembacken, O. LeMoine, K. Kuznetsov, J.-F. Rey, T. Rösch, G. Eisen, P. Cotton, and M. Fujino, “Minimal standard terminology for gastrointestinal endoscopy—MST 3.0,” Organization Mondiale Endoscopia Digestive, Committee for Standardization and Terminology, 2008, incorporated herein by reference), which offers a standardized selection of terms and attributes for the description of findings, procedures, and complications. The current release of the minimal standard terminology includes 26 reasons, 7 complications, 30 diagnoses, 3 examinations, 38 findings, 15 sites, and 8 additional diagnostic procedures relevant for a colonoscopic video interpretation system.

In addition to these clinical features, the video interpretation system is preferably augmented by taking into account features related to frame degradation factors, such as obstructions, blur, glare, and illumination, objects in the colonoscopic video scene such as blood, stool, water, and surgical tools, and descriptive findings such as color, edges, boundaries, and regions. Obstructions can be any object in the colonoscopic video scene that degrade or block the view and, as such, do not hold any useful information about the underlying tissues. Degraded frames are detected and excluded in order to reduce the computational burden and improve the performance of the video interpretation system.

Further, the design of the system is flexible in that additional relationship dimensions can be applied to any colonoscopic features visible in the colonoscopic video scenes and, as such, increase the training data set and further improve the performance of the video interpretation system.

The system can optionally take frames and segments (relevant sections) labeled by the output from feature detection algorithms as input, further increasing its capabilities.

In other embodiments, the video interpretation system can be applied to other types of video data, including but not limited to other endoscopic procedures such as upper endoscopy, enteroscopy, bronchoscopy, endoscopic retrograde cholangiopancreatography, and augment or change the feature sets accordingly. Non-medical applications include such applications as surveillance, automatic driving, robotic vision, summary of news broadcast extracting the main points, automatic video tagging for online videos and pipeline examination, for example.

### Preprocessing

A set of pre-processing steps is preferably applied prior to the SSEHMM, in order to calibrate and improve the quality of the video data, and to detect glare regions, edges and potential tissue boundaries.

For endoscopic video data that typically exhibits so-called barrel-type spatial distortion caused by the wide angle design of the optics, distortion correction can be applied (for example, as described in W. Li, S. Nie, M. Soto-Thompson, and Y. I. A-Rahim, “Robust distortion correction of endoscope,” Proc. SPIE 6819, pp. 691812-1—8, 2008, incorporated herein by reference).

For standard video data, de-interlacing can be applied in order to remove any distortion and interlacing artifacts that otherwise could obscure the true feature information. Other video quality enhancements that can be applied include, but are not limited to, noise reduction, contrast enhancement, super resolution (a method to use multiple video frames of the same object to achieve a higher resolution image) and video stabilization (such as described in a co-pending, commonly assigned U.S. patent application Ser. No. 11/895,150 for “Computer aided diagnosis using video from endoscopes,” filed Aug. 21, 2006; and EP patent no. 2054852 B1. “Computer aided diagnosis using video from endoscopes,” incorporated herein by reference).

Glare could be identified by detecting saturated areas and small high contrast regions (for example, as described in H. Lange, “Automatic glare removal in reflectance imagery of the uterine cervix,” Proc. SPIE 5747, pp. 2183-2192, 2005, incorporated herein by reference). Edges are also detected, preferably using a Sobel edge filter (R. C. Gonzales and R. E., Digital image processing, Second Edition, Upper Saddle River, Prentice-Hall, 2002, incorporated herein by reference), but other methods providing similar results can also be used. The detected edges can then be linked to their nearest neighbors using an edge linking algorithm (for example, as described in Q. Zhu, M. Payne, and V. Riordan, “Edge linking by directional potential function (DPF),” Image and Vision Computing 14(1), pp. 59-70, 1996, incorporated herein by reference). Potential tissue boundaries can then be identified based on the edge curvature (for example, as described in Q. Zhu, M. Payne, and V. Riordan, “Edge linking by directional potential function (DPF),” Image and Vision Computing 14(1), pp. 59-70, 1996, incorporated herein by reference), and candidate colon tissue regions of interest can be extracted from each frame for input to the SSEHMM model.

In the training phase of the HMM, an eigentissue approach is preferably used to describe the characteristics of the different features in the colonoscopic video. First, training image windows, which are subsets of an entire video frame from different angles of the different features present in the endoscopic video, are extracted. Then, with M training image windows and l features, a set of vectors Γⁱ representing the training image windows for feature i is defined as

Γⁱ={r₁ⁱ, Γ₂ⁱ, Γ₃ⁱ, K, Γ_{M}ⁱ} (1)

where Γₘⁱ represents the m-th training image vector for feature i. After obtaining the training image set Γⁱ, the mean image vector Ψⁱ for each feature i is generated as

$${Ψ}^{i} = \frac{1}{M} {\sum }_{m = 1}^{M} {Γ}_{m}^{i} . \qquad ( 2 )$$

The covariance matrix Cⁱ is then determined according to

$${C}^{i} = \frac{1}{M} {\sum }_{m = 1}^{M} ( {Γ}_{m}^{i} - {Ψ}^{i} ) \cdot {( {Γ}_{m}^{i} - {Ψ}^{i} )}^{T} \qquad ( 3 )$$

and the M eigenvectors v₁ⁱ, v₂ⁱ, v₃ⁱ, K, v_{M}ⁱ of the covariance matrix are computed to define a set of eigentissues for feature group i. This means that the eigentissue space is defined as the space spanned with eigenvectors of the covariance matrix of the training video segments (relevant sections).

A feature space for feature group i is also defined as the space spanned by the eigentissues. That is, each feature image can be represented as a linear combination of the eigentissues. Since the magnitude of the eigenvalue represents how much the corresponding eigentissue characterizes the variance between the images, M′ diagnostically relevant eigentissues can be extracted from the original M eigentissues, with M′<M, by selecting the eigentissues with the highest eigenvalues. Therefore, the dimension of the feature space can be reduced from M to M′ and any feature image window can be represented by an M′-dimensional score vector in the reduced dimension feature space.

As the last step, a feature score is defined for the different colon features as the Euclidean distance (the distance between pairs of points in Euclidean space) between the score vector of a feature image window and the eigentissues in the feature space. For each tissue image window, there are M′ feature scores for each feature and, therefore, I×M′ feature scores for all windows.

### First Level HMM for Intra-Frame Relationships

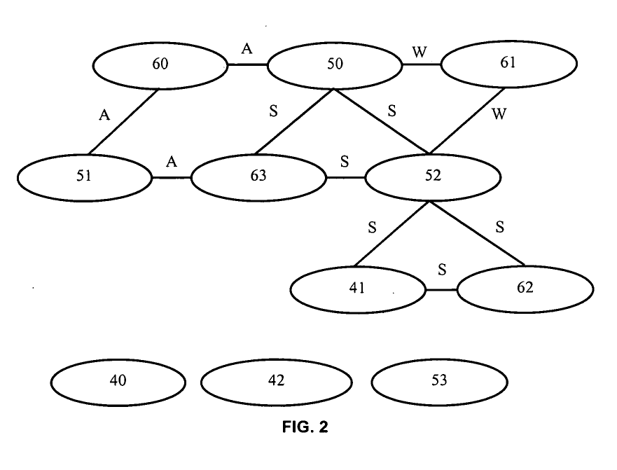

Based on observations from colonoscopy video data, FIG. 2 shows the relationships between the colonoscopic features of blur (40), glare (41), illumination (42), blood (50), stool (51), surgical tools (52), water (53), diverticula (60), mucosa (61), lumen (62), and polyps (63). The relationships represent the likelihood of observing the two features in the same video frame or in subsequent video frames during a relatively short time period. Strong (S) relationships can be identified between polyps (63), lumen (62), glare (41), blood (50) and surgical tools (52) while a weak (W) relationship can be observed between mucosa (61), blood (50) and surgical tools (52). Average (A) relationships can be seen between polyps (63), diverticula (60), and stool (51). No significant relationships can be deduced for blur (40), illumination (42), and water (53).

In order to model the dependencies between the different features, a region-based approach is applied to identify the features in a colonoscopic video frame. Let fⱼⁱ denote the jth frame of the ith video; then, frame fⱼⁱ is composed of K disjoint image regions such that

$${f}_{j}^{i} = {Y}_{k}^{K} {r}_{j , k}^{i} , \qquad ( 4 )$$

where r_{j,k}ⁱ represents the k^{th} region of the jth frame of the ith video and r_{j,k}ⁱ I r_{j,l}ⁱ=φ for k≠l.

Then, the neighborhood ∂_{j,k}ⁱ of region r_{j,k}ⁱ is defined as the set of regions adjacent to the region r_{j,k}ⁱ. Following the stochastic HMM framework, a hidden state s_{j,k}ⁱ of region r_{j,k}ⁱ is defined as representing whether or not features areₖ contained in region r_{j,k}ⁱ. Based on this, the number of possible states will be 2^{N}^{ₒ}, where Nₒ is the number of features. An observation o_{j,k}ⁱ in region r_{j,k}ⁱ is defined by the image clip corresponding to region r_{j,k}ⁱ. Finally, the random variables S_{j,k}ⁱ and O_{j,k}ⁱ are defined to represent state s_{j,k}ⁱ and observation o_{j,k}ⁱ, respectively. The Markov property makes the following hold for each state s_{j,k}ⁱ, k=1, Λ, K

p(s_{j,k}ⁱ|S_{j,l}ⁱ,Λ,S_{j,k-1}ⁱ,S_{j,k+1}ⁱ,Λ,S_{j,K}ⁱ,O_{j,k}ⁱ)=p(s_{j,k}ⁱ|N_{j,k}ⁱ,O_{j,k}ⁱ), (5)

where p represents the conditional probability density function of the state and N_{j,k}ⁱ denotes the set of the neighbor states of s_{j,k}ⁱ such that N_{j,k}ⁱ={s_{j,l}ⁱ|r_{j,l}ⁱε∂_{j,k}ⁱ}.

Following the Hammersley-Clifford theorem (P.L. Dobrushin, “The description of a random field by means of conditional probabilities and conditions of its regularity,” Theory of Probability and its Applications 13(2), pp. 197-224, 1968, incorporated herein by reference), the joint conditional probability density function p(sⱼⁱ|Oⱼⁱ=oⱼⁱ) can be written as

$$p ( {s}_{j}^{i} \vert {O}_{j}^{i} = {o}_{j}^{i} ) = \frac{1}{Z ( {o}_{j}^{i} )} \cdot exp ( {\sum }_{s ∈ {N}_{j , k}^{i} , k = 1}^{k = K} λ \cdot t ( s , {s}_{j , k}^{i} , {o}_{j , k}^{i} ) + {\sum }_{k = 1}^{K} μ \cdot u ( {s}_{j , k}^{i} , {o}_{j , k}^{i} ) ) , \qquad ( 6 )$$

where sⱼⁱ={s_{j,k}ⁱ|k=1, Λ, K}, Oⱼⁱ={O_{j,k}ⁱ|k=1, Λ, K}, oⱼⁱ={o_{j,k}ⁱ|k=1, Λ, K}, t is a transition feature function, u is a state feature function, λ and μ are parameters to be estimated, and Z(oⱼⁱ) is a normalization factor such that

$$Z ( {o}_{j}^{i} ) = {\sum }_{{s}_{j}^{i}} [ exp ( {\sum }_{s ∈ {N}_{j , k}^{i} , k = 1}^{k = K} λ \cdot t ( s , {s}_{j , k}^{i} , {o}_{j , k}^{i} ) + {\sum }_{k = 1}^{K} μ \cdot u ( {s}_{j , k}^{i} , {o}_{j , k}^{i} ) ) ] , \qquad ( 7 )$$

An important design issue in the disclosed system is to determine the state feature function u and the transition feature function t in Equation (6). In particular, determining the transition feature function t is of interest since this function captures the relationship between features in neighboring regions.

### Second Level HMM for Inter-Frame Relationships

The first level HMM for intra-frame relationships yields the conditional probability density function p(sⱼⁱ|Oⱼⁱ=oⱼⁱ) for each frame j in the ith video as in Equation (6). For the second level HMM for inter-frame relationships a frame-wise feature appearance, ôⱼⁱ, is defined according to

$${\hat{o}}_{j}^{i} = {argmax}_{{s}_{j}^{i}} p ( {s}_{j}^{i} \vert {O}_{j}^{i} = {o}_{j}^{i} ) . \qquad ( 8 )$$

This frame-wise appearance, ôⱼⁱ, is referred to as a pseudo-observation of frame j since it treated as an observation in the second-level HMM model and Ôⱼⁱ denotes the corresponding random variable.

The variables tⱼⁱ and by Tⱼⁱ are defined as the hidden state variable and the corresponding random variable of the jth frame of ith video. This hierarchical two level approach connects the intra-frame relationships in a first level HMM with the inter-frame relationships in a second level HMM.

This approach is novel and different from any published HMM-based approaches in video data analysis, which only consider spatial and temporal relationships independently, including hierarchical HMMs (L. Xie, S.F. Chang, A.

Divakaram, and H. Sun, “Unsupervised discovery of multilevel statistical video structures using hierarchical hidden Markov models,” Proc. 2003 International Conference on Multimedia and Expo (ICME'03), 2003, incorporated herein by reference) and multi-dimensional HMMs (as discussed in J. Jiten, Multidimensional hidden Markov model applied to image and video analysis, PhD Thesis, Telecom ParisTech (ENST), 2007, and J. Jiten and B. Merialdo, “Video modeling using 3-D hidden Markov model,” Proc. Second International Conference on Computer Vision and Applications, 2007, incorporated herein by reference).

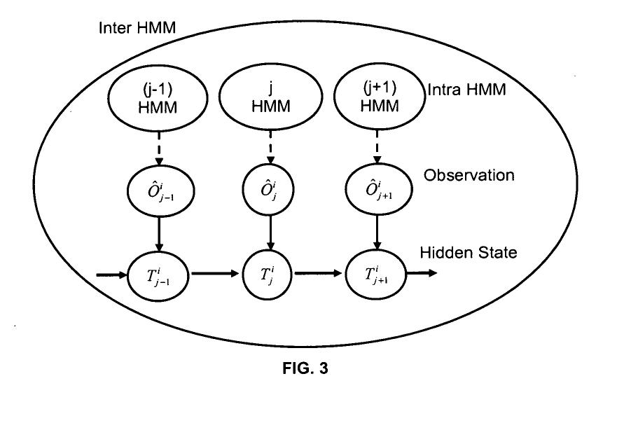

The structure of the two-level HMM for intra- and inter-frame relationships is depicted in FIG. 3 and mathematically the Markov property of the two-level HMM is represented as

p(tⱼⁱ|T₁ⁱ, Λ, Tⱼ₋₁ⁱ, Ôⱼⁱ)=p(tⱼⁱ|Tⱼ₋₁ⁱ,Ôⱼⁱ), ∀j=2, Λ, Jᵢ, (9)

where p represents the conditional probability density function of the state and Jᵢ is the number of frames in the ith video.

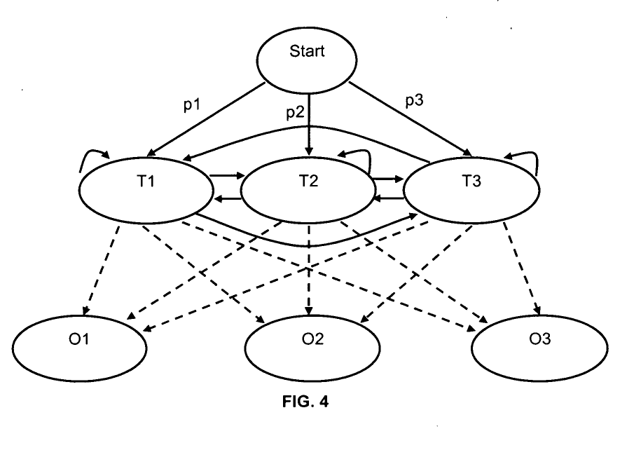

The probabilistic relationship of state transitions and observations are illustrated in FIG. 4 with T1, T2 and T3 depicting three state transitions between, for example, a polyp, diverticula, and mucosa, and O1, O2, and O3 depicting the observations of features in the video data such as polyp with blood, blood only, and diverticula with stool, and p1, p2, and p3 being the conditional probabilities of observing the features in the training dataset.

The transition probabilities aₘₙ representing the probability of transitioning from state m to state n are defined as

aₘₙ=p(tⱼⁱ=n|tⱼ₋₁ⁱ=m), (10)

where m, nεΣ and Σ, |Σ|=2^{N}^{ₒ}, is the set of possible states. Furthermore, the observation probabilities bₘₗ representing the probability that the pseudo-observation is l when the state is m are in turn defined as

bₘₗ=p(ôⱼⁱ=l|tⱼⁱ=m), (11)

where mεΣ, lεΩ, and Ω is the set of possible observations.

### Embedded HMM for Data Quality, Anatomical Structures, and Multimodality

The preferred embodiment of the tissue interpretation system contains embedded models to consider video quality, anatomical structures, and multimodality video data. An embedded HMM (EHMM) is a generalized HMM with a set of so-called superstates, each of which is itself an HMM. This embedding concept is preferably applied in a hierarchical manner by first modeling the video quality, then the anatomical structures and finally the multi-modality of the video data. This hierarchical scheme provides an explicit modeling of the multi-dimensional nature of the data and, furthermore, significantly reduces the computational complexity of the tissue interpretation system.

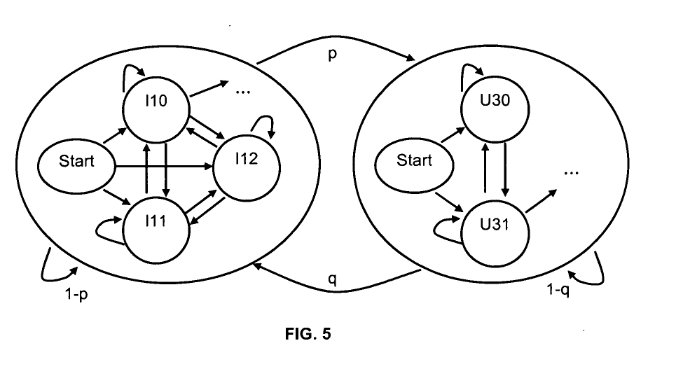

Colonoscopy videos are composed of informative video frames from which we can extract clinical information and uninformative (or featureless) video frames that do not contain any useful information. The video quality EHMM is therefore modeled as informative and uninformative superstates. The informative superstate is modeled as the two-level HMM described above. The uninformative superstate is modeled as two-level HMM, but with a different set of second level states including “artifacts” such as frame degradation factors, objects and “motion blur” caused by the movement of the colonoscope or the colon. FIG. 5 illustrates the structure and the probabilistic state transition of the data quality EHMM with I10, I11, and I12 depicting different informative states (such as diverticula, polyp, and mucosa), U30 and U31 depicting uninformative states (such as artifacts and motion blur), and p and q being the state transition probabilities from ‘informative to uninformative’ and ‘uninformative to informative’, respectively.

For the data quality EHMM, a combination of two quantitative measures is preferably used for assessing video frame quality: Shannon's entropy (C. E. Shannon, “A mathematical theory of communication,” ACM SIGMOBILE Mobile Computing and Communications Review 5(1), 3-55, 2001, incorporated herein by reference) and Range filter (C. Tomasi and R. Manduchi, Bilateral filtering for gray and color images, Proc. Sixth International Conference on Computer Vision (ICCV'98), pp. 839-846, 1998, incorporated herein by reference).

The first measure, Shannon's entropy H(A), represents the amount of the information contained in an image, and is defined as

H(A)=−Σp(a)log₂ p(a) (12)

where A is a random variable representing pixel intensity, and a is a realization of A. The probability mass function of A is denoted A(•). The second measure, range filter R(Ω), is the mean of the range-filtered values of an image and is defined as

$$R ( Ω ) = \frac{{\sum }_{i ∈ Ω} {max}_{j , k ∈ {N}_{i}} ( {I}_{j} - {I}_{k} )}{n} \qquad ( 13 )$$

where Ω is the set of the pixels in the image, Nᵢ is the set of pixels in the window centered around the pixel i, lⱼ and Iₖ are the intensities of pixel j, and k, respectively, and n is the total number of pixels in the image.

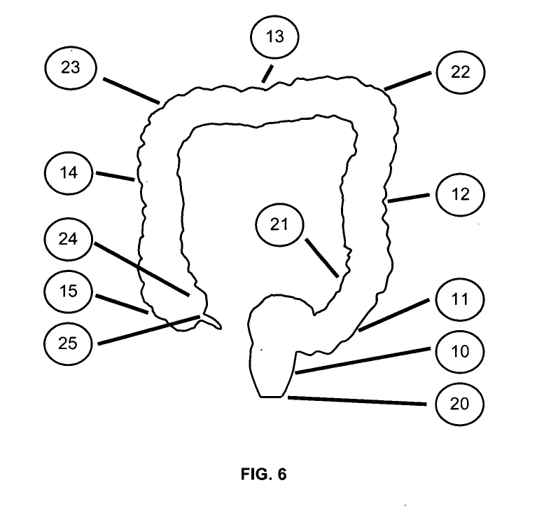

In order to account for different anatomical structures in the video frames, another embedding is applied. The colon, as illustrated in FIG. 6, consists of six anatomical segments: rectum (10), sigmoid colon (11), descending colon (12), transverse colon (13), ascending colon (14), and cecum (15). The anatomical EHMM models these segments as another set of six superstates. The transitions between the different anatomical segments in the colon are preferably inferred by the use of anatomical landmarks (see FIG. 6) such as the anus (20), sigmoid/descending colon transition (21), splenic flexure (22), hepatic flexure (23), ileocecal valve (24), and appendiceal orifice (25).

Different imaging modalities are modeled using a top-level EHMM with superstates representing each imaging modality. Colonoscopy typically employs four imaging modalities: white light reflectance, narrow-band reflectance, fluorescence, and chromo-endoscopy. Therefore, the imaging modality EHMM contains at least four superstates representing these four modalities. Each of the four superstates contains separate embedded EHMMs governing transitions between low and high quality video frames and the anatomical structures of the colon. Transitions between the four imaging modality superstates occur when the physician changes between imaging modalities.

### Forward Backward Algorithm

The most probable classification for each frame, in a video is preferably determined using the forward-backward algorithm (for example as described in K. Tokuda, T. Yoshimura, T. Masuko, T. Kobayashi, and T. Kitamnura, “Speech parameter generation algorithms for HMM-based speech synthesis,” Proc. IEEE International Conference on Acoustics, Speech, and Signal Processing (ICASSP'00), pp. 1315-1318, 2000; and J. Lafferty, A. McCallum, and F. Pereira, “Conditional random fields: probabilistic models for segmenting and labeling sequence data,” Proc. Eighteenth International Conference on Machine Learning, pp. 282-289, 2001, incorporated herein by reference). The forward-backward algorithm is an efficient method for calculating the probability of a state sequence given a particular observation sequence. The most likely state sequence, as determined by the algorithm, is selected as the interpretation of the given video frames.

### Model Parameter Estimation

To estimate the parameters λ and μ, of the joint conditional probability density function p(sⱼⁱ|Oⱼⁱ=oⱼⁱ) in Equation (6), a maximum likelihood estimation (for example as described in L.J. Cox, S.L. Hingorani, S.B. Rao, and B.M. Maggs, “A maximum likelihood stereo algorithm”, Computer Vision and Image Understanding 63(3), pp. 542-567, 1996, incorporated herein by reference), a common principle for parameter estimation in the HMM framework, is preferably applied. However, other parameter estimation methods providing similar results can also be used. The method starts by defining a log-likelihood function L(λ, μ) as

$$L ( λ , υ ) = {\sum }_{d = 1}^{D} [ {\sum }_{j = 1}^{J} log \frac{1}{Z ( {o}_{j}^{d} )} + {\sum }_{j = 2}^{J} {\sum }_{k = 1}^{K} λ \cdot t ( {s}_{j - 1 , k}^{d} , {s}_{j , k}^{d} , {o}_{j , k}^{d} ) + {\sum }_{j = 1}^{J} {\sum }_{k = 1}^{K} μ \cdot u ( {s}_{j , k}^{d} , {o}_{j , k}^{d} ) ] \qquad ( 14 )$$

where D is the number of training state sequences and superscript d means that the superscripted variable corresponds to the d-th state sequence. By maximum likelihood, the estimated parameters λ^{ML} and μ^{ML} are obtained by

$$( {λ}^{ML} , {μ}^{ML} ) = {argmax}_{( λ , u )} L ( λ , μ ) \qquad ( 15 )$$

As displayed by Equation (15), the parameter estimation requires nonlinear optimization; the Newton-Raphson method (which is a method of finding successively better approximations to roots of a function) is widely used for this purpose. However, the Newton-Raphson method involves computing and iteratively updating the so-called Hessian matrix (which is the second-order partial derivatives of a function and, as such, describes the local curvature of the function) of the likelihood function, which is difficult if the likelihood function is complex, as it is in this case. In order to avoid this complication, the current invention adopts a quasi-Newton method in which the Hessian matrix does not need to be computed analytically. The particular application of this method to the maximum likelihood estimation is described as follows.

First, define θ to be a parameter vector including λ and μ such that θ=[λ^{T}μ^{T}]^{T}. Then, the gradient ∇L(θ) of the likelihood function L(θ) is represented as

$$∇ L ( θ ) = {[ \frac{\partial L ( θ )}{\partial {θ}_{l}} , Λ , \frac{\partial L ( θ )}{\partial {θ}_{J}} ]}^{T} \qquad ( 16 )$$

where J is the total number of parameters, J_{A} is the number of parameters in λ, and

$$\begin{matrix}\frac{\partial L ( θ )}{\partial {θ}_{l}} = \{ \begin{matrix}{\sum }_{d = 1}^{D} [ {\sum }_{i = 2}^{n} {t}_{l} ( {s}_{i - 1}^{d} , {s}_{i}^{d} , {o}^{d} ) - \frac{{W}_{1 , l} ( {o}^{d} )}{Z ( {o}^{d} )} ] , & if l ≧ {J}_{λ} , \\ {\sum }_{d = 1}^{D} [ {\sum }_{i = 1}^{n} {u}_{l} ( {s}_{i}^{d} , {o}^{m} ) - \frac{{W}_{2 , l} ( {o}^{m} )}{Z ( {o}^{m} )} ] , & if {J}_{λ} < l ≦ J ,\end{matrix} \\ and & ( 17 ) \\ {W}_{1 , l} ( {o}^{d} ) = {\sum }_{s ε {Ω}_{λ}} [ {\sum }_{i = 2}^{n} {t}_{l} ( {s}_{i - 1} , {s}_{i} , {o}^{d} ) \cdot exp ( {\sum }_{i = 2}^{n} {\sum }_{l} {λ}_{l} {t}_{l} ( {s}_{i - 1} , {s}_{i} , {o}^{d} ) + {\sum }_{i = 1}^{n} {\sum }_{l} {μ}_{l} {u}_{l} ( {s}_{i} , {o}^{d} ) ) ] & ( 18 )\end{matrix}$$

Then, maximum likelihood parameter estimation θ^{ML} is determined by iteratively updating θ such that

θ(k+1)=θ^{(k)}+α^{(k)}d^{(k)} (19)

and

d^{(k)}=D^{(k)}∇L(θ^{(k)}) (20)

where the superscripts in parentheses represent the iteration, d^{(k)} is the update direction of k-th iteration, α^{(k)} is the step size of k-th iteration, and D^{(k)} is a positive definite matrix, which may be adjusted from one iteration to the next so that the direction d^{(k)} tends to approximate the Newton direction. D^{(k)} is preferably obtained using the Broyden-Fletcher-Goldfarb-Shannon (BFGS) method as

$${D}^{( k )} = {D}^{( k - 1 )} + \frac{( {θ}^{( k )} - {θ}^{( k - 1 )} ) {( {θ}^{( k )} - {θ}^{( k - 1 )} )}^{T}}{{( {θ}^{( k )} - {θ}^{( k - 1 )} )}^{T} ( ∇ L ( {θ}^{( k )} ) - ∇ L ( {θ}^{( k - 1 )} ) )} - \frac{{D}^{( k - 1 )} ( ∇ L ( {θ}^{( k )} ) - ∇ L ( {θ}^{( k - 1 )} ) ) {( ∇ L ( {θ}^{( k )} ) - ∇ L ( {θ}^{( k - 1 )} ) )}^{T} {D}^{( k - 1 )}}{{( ∇ L ( {θ}^{( k )} ) - ∇ L ( {θ}^{( k - 1 )} ) )}^{T} {D}^{( k - 1 )} ( ∇ L ( {θ}^{( k )} ) - ∇ L ( {θ}^{( k - 1 )} ) )} + \\ {( ∇ L ( {θ}^{( k )} ) - ∇ L ( {θ}^{( k - 1 )} ) )}^{T} {D}^{( k - 1 )} ( ∇ L ( {θ}^{( k )} ) - ∇ L ( {θ}^{( k - 1 )} ) ) {{υ}^{( k - 1 )} ( {υ}^{( k - 1 )} )}^{T} , \qquad ( 21 )$$

where k>1 and

$${υ}^{( k )} = \frac{( {θ}^{( k + 1 )} - {θ}^{( k )} )}{{( {θ}^{( k + 1 l )} - {θ}^{( k )} )}^{T} ( ∇ L ( {θ}^{( k + 1 )} ) - ∇ L ( {θ}^{( k )} ) )} - \frac{{D}^{( k )} ( ∇ L ( {θ}^{( k + 1 )} ) - ∇ L ( {θ}^{( k )} ) )}{{( ∇ L ( {θ}^{( k + 1 )} ) - ∇ L ( {θ}^{( k )} ) )}^{T} {D}^{( k )} ( ∇ L ( {θ}^{( k + 1 )} ) - ∇ L ( {θ}^{( k )} ) )} . \qquad ( 22 )$$

The initial D⁽¹⁾ is an arbitrary symmetric positive definite matrix which is usually the identity matrix.

Now, consider the expression of the component

$$\frac{\partial L ( θ )}{\partial {θ}_{l}}$$

of the gradient ∇L(θ).

The first term of the expression associated with either tₗ(sᵢ₋₁^{d},sᵢ^{d},o^{d}) or uₗ(sᵢ^{d},o^{d}) is straightforward and easy to compute since it is evaluated for the fixed training data. However, the second term requires complicated computation, in particular, for cases where the set of possible sequences s, Ωₛ, is very large. One efficient way to compute this term is by matrix multiplication. First, consider the computation of z(o^{d}). Let K be the number of possible states for any sᵢ. Then, the K×K matrix M^{Z} is defined by

$${M}_{i , j}^{Z} = exp ( {\sum }_{l} {λ}_{l} {t}_{l} ( {s}_{i} , {s}_{j} , {o}^{d} ) + {\sum }_{l} {μ}_{l} {u}_{l} ( {s}_{j} , {o}^{d} ) ) \qquad ( 23 )$$

where m_{i,j}^{Z} is the (i,j)-th element of M^{Z}. Using matrix multiplication, Z(o^{d}) is computed as

Z(o^{d})=[1,K,l](M^{Z})ⁿ[1,K,l]^{T} (24)

Similarly, K×K matrices M^{W1,l} and M^{W2,l}, l=1, . . . , J for computing W_{1,l}(o^{d}) and W_{2,l}(o^{d}), respectively, are:

$$\begin{matrix}{M}_{i , j}^{W 1 , l} = {t}_{l} ( {s}_{i} , {s}_{j} , {o}^{d} ) exp ( {\sum }_{l} {λ}_{l} {t}_{l} ( {s}_{i} , {s}_{j} , {o}^{d} ) + {\sum }_{l} {μ}_{l} {u}_{l} ( {s}_{j} , {o}^{d} ) ) \\ and \\ & ( 25 ) \\ {M}_{i , j}^{W 2 , l} = {μ}_{l} ( {s}_{j} , {o}^{d} ) exp ( {\sum }_{l} {λ}_{l} {t}_{l} ( {s}_{i} , {s}_{j} , {o}^{d} ) + {\sum }_{l} {μ}_{l} {u}_{l} ( {s}_{j} , {o}^{d} ) ) & ( 26 )\end{matrix}$$

where M_{i,j}^{W1,l} and M_{i,j}^{W2,l} are the (i,j)-th elements of M^{W1,l} and of M^{W2,l}, respectively. Then, W_{1,l}(x^{m}) and W_{2,l}(x^{m}) are computed as

W_{1,l}(x^{m})=[1,Λ,l](M^{W1,l})ⁿ[1,Λ,l]^{T} (27)

and

W_{2,l}(x^{m})=[1,Λ,l](M^{W2,l})ⁿ[1,Λ,l]^{T} (28)

Furthermore, in order to enhance the performance of the method, the preferred embodiment of the video interpretation system designs the quasi-Newton method with inner and outer iterations. That is, each outer iteration is composed of J inner iterations, and, when the next outer iteration starts, the starting D^{(k)} is reset as the initial D^{(l)}. This restarting scheme prevents the Hessian approximation D^{(k)} from becoming indefinite or singular due to reasons such as modeling error for quadratic approximation, inexact line search for α^{(k)}, and computational rounding errors.

### Physician Feedback

Feedback from physicians (colonoscopist) is important in improving the accuracy of the video interpretation system. Physician feedback can take many forms. One form is to provide input regarding the quality of the video frames (informative versus uninformative) as simple “true” or “false” statements. A second form is to input the colon landmarks (such as the anus (20), sigmoid/descending colon transition (21), splenic flexure (22), hepatic flexure (23), ileocecal valve (24) and appendiceal orifice (25) as illustrated in FIG. 6) and colon segments (rectum (10), sigmoid colon (11), descending colon (12), transverse colon (13), ascending colon (14), and cecum (15) as illustrated in FIG. 6) in the colon video. A third form is to assess the accuracy of the classifications and annotations as “true” or “false” statements for the entire video frame, or as a conditional “true” statement meaning that the feature is present in the video frame, but at an incorrect location.

To facilitate complex interaction with physicians, the video interpretation system preferably includes a graphical user interface which allows the users to efficiently query and retrieve video frames of interest with flexible search criteria. Furthermore, the system would preferably enable users to review and modify retrieved video frame classifications and annotations. The video frames for which annotations have been reviewed and modified are then used for semi-supervised learning for the un-reviewed video frames.

### Semi-Supervised Learning

Most of the popular learning schemes for HMMs are inductive; that is, the model parameters are estimated using training data only. However, inductive learning yields undesirable biases if the training set does not represent general data properly. This limits the usefulness of inductive learning when applied to colonoscopy video interpretation because the videos show considerable variation between patients and procedures. Semi-supervised learning can alleviate this bias by training with both labeled and unlabeled video. The direct involvement of test data in the learning process increases the estimated model's generalization capability. Moreover, expertly annotated or interpreted video data is expensive, while raw video is widely available.

In the preferred embodiment of the video interpretation system, the expectation maximization (EM) algorithm (Y. Wu and T.S. Huang, “Color tracking by transductive learning,” Proc. IEEE Conference Computer Vision and Pattern Recognition (CVPR'00), pp. 133-138, 2000, incorporated herein by reference) is used as the semi-supervised learning scheme. Other methods providing similar results can also be used. Assume N colonoscopy videos are available, of which N₁ include annotations and (N−N₁) do not. Denote by Dₗ and by D_{∪} the colonoscopy video data sets with and without interpretations, respectively, such that Dₗ={vⁱ,wⁱ}ᵢ₌₁^{N}^{ₗ}: and D_{U}={vⁱ,ŵⁱ}_{i=N}_{ₗ}₊₁^{N}, where vⁱ is the i^{th} video, wⁱ is the expert's interpretation for the i^{th} video, and ŵⁱ is the unknown interpretation for the i^{th} video. Now, denote obj(D;Θ)) as the objective function to be maximized for the EHMM parameter estimation, where D is a data set and Θ is the model parameter set. This objective function is defined by the probability of the state sequence of the EH MM which is derived from the forward-backward algorithm. Then, the (q+1)th step of the EM algorithm is designed as

$${( \hat{w} )}^{q + 1} = {arg max}_{\hat{w}} obj ( D ( \hat{w} ) ; {( Θ )}^{q} . ) \qquad ( 29 )$$

for the expectation (E) step, and

$${( Θ )}^{q + 1} = {arg max}_{Θ} obj ( {D ( \hat{w} )}^{q + 1} ; Θ ) \qquad ( 30 )$$

for the maximization (M) step with ŵ={ŵⁱ}_{i=N}_{ₗ}₊₁^{N} being the set of the unknown interpretations. The E-Step of Equation (29) updates the unknown interpretations with the model parameters determined at the previous M-step, and the M-Step of Equation (30) updates the model parameters with the updated interpretations determined at the previous E-step. These updates are iterated until the algorithm converges.

### Video Visualization and Management System

A clinical data visualization and management system provides physicians and users with a set of tools, functions, and systems during and after the course of colonoscopic exams. In the context of the disclosed invention, the video visualization and management system would, in addition to the live video available during an exam, provide at least the following

(1) capture, storage, search, and retrieval of all patient, exam, and video information

(2) image enhancement technologies that improve visualization,

(3) a generic digital colon model that enables visual navigation through colon videos,

(4) a feature alert system which automatically interprets the colon video and classifies and annotates the findings,

(5) a screening system which detects and tracks the diagnostically important features of polyps and diverticula,

(6) a segmentation (filtering) method which filters colon exam videos into clinically relevant or irrelevant segments (sections),

(7) a synchronization method of exam videos for longitudinal exam comparisons,

(8) a field of view scoring system that assess the completeness of the exam

### Storage, Capture, Search, and Retrieval

During the course of colonoscopic exams, the live video data is preferably captured and stored in either local or remote disk storage. In order to provide efficient search and retrieval functions for the video data, a relational database is preferably utilized. To index and improve the database efficiency, the content-based properties of the video data are preferably used. For retrieval, two main search functions are preferably used.

Keyword Search allows for keyword searches related to the minimal standard terminology for endoscopy and other features, including but not limited to, frame degradation factors (such as featureless, blur, glare, and illumination), objects in the colonoscopic video scene (such as blood, stool, water, and tools) patient information (such as age and gender) and video information (such as video file, segment (relevant section), and frame numbers).

Index Search allows for fast and efficient data retrieval. This search is preferably based on a semantic indexing scheme that allows users to relate colonoscopic features, within and between video frames, using correlation measures. This search function also provides support for a quality control index which indicates diagnostically informative frames only. Any frame which is not qualified for diagnostic support is subsequently not considered for further semantic imaging. Furthermore, patient follow-up indexing is preferably included to support physician's clinical judgment for re-examination.

### Image Enhancement

Image enhancement can be applied to the colonoscopic video data, both during and after an exam, in an effort to improve the quality of the data or enhance clinically relevant features such as vessel structures, tissue structures and lesion borders. Different image enhancement methods can be applied including, but not limited to, noise reduction, contrast enhancement, super resolution, and video stabilization (such as described in the co-pending, commonly assigned U.S. patent application Ser. No. 11/895,150 for “Computer aided diagnosis using video from endoscopes”, filed Aug. 21, 20061 and EP patent no. 2054852 B1 “Computer aided diagnosis using video from endoscopes,” incorporated herein by reference). In addition, the image enhancement can include calibration and correction methods, such as color calibration to ensure that the color is identical for every exam video, and distortion correction to ensure that the features are correctly displayed, irrespective of the instrument used to collect the data (for example, utilizing methods described in W. Li, M. Soto-Thompson, U. Gustafsson, “A new image calibration system in digital colposcopy,” Optics Express 14 (26), pp. 12887-12901, 2006; and W. Li, S. Nie, M. Soto-Thompson, and Y. I. A-Rahim, “Robust distortion correction of endoscope,” Proc. SPIE 6819, pp. 691812-1—8, 2008, incorporated herein by reference).

### Digital Colon Model

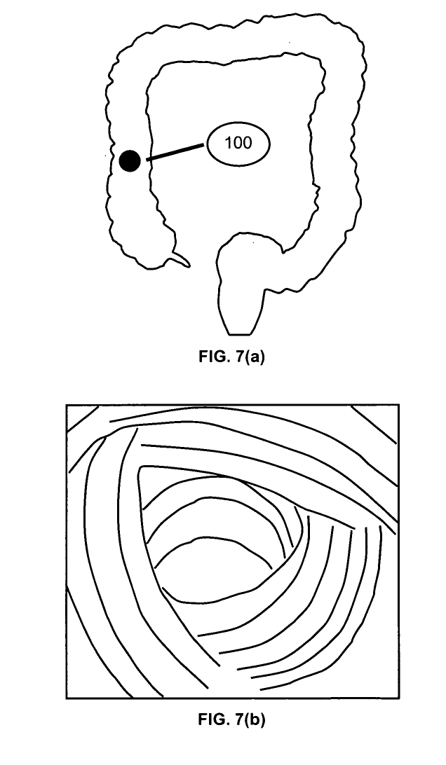

A digital colon model is a visualization tool that enables standardized navigation through colon videos, as illustrated in FIG. 7. Starting with a generic colon model as illustrated in FIG. 7(a) (preferably, as illustrated in FIG. 6, consisting of the five anatomical colon segments of the rectum (10), sigmoid colon (11), descending colon (12), transverse colon (13), ascending colon (14), and cecum (15), and anchored by the anatomical colon landmarks of the anus (20) sigmoid/descending colon transition (21), splenic flexure (22), hepatic flexure (23), and ileocecal valve (24)) the video data as illustrated in FIG. 7(b) are mapped and superimposed onto the geometry of this generic model. While viewing the video data, either in real-time during a clinical exam or as part of a video review, an icon in the colon model (see FIG. 7(a)) depicts the estimated location of the colonoscope tip (100) within the colon.

This digital colon model is a standardized visualization tool for colonoscopy because every exam video can be superimposed onto the generic colon model. Furthermore, the digital colon model can help the physician to plan their treatment during the examination of the colon. For example, during entry, the physician can mark suspicious locations on the digital colon model. During withdrawal, the physician can be alerted to previously digitally marked regions and perform treatment. Additionally, for high-risk patients that require surveillance, the model can provide a framework for registering the patient's clinical state across exams, thereby enabling change detection.

The concept of the digital colon model can be augmented by, and in addition to, video data acquired using different macroscopic imaging modalities, including data from microscopic and spectroscopic probe systems, such as confocal microscopy, optical coherence tomography, and infrared spectroscopy. These technologies provide imaging or spectral information about the tissue on a microscopic scale.

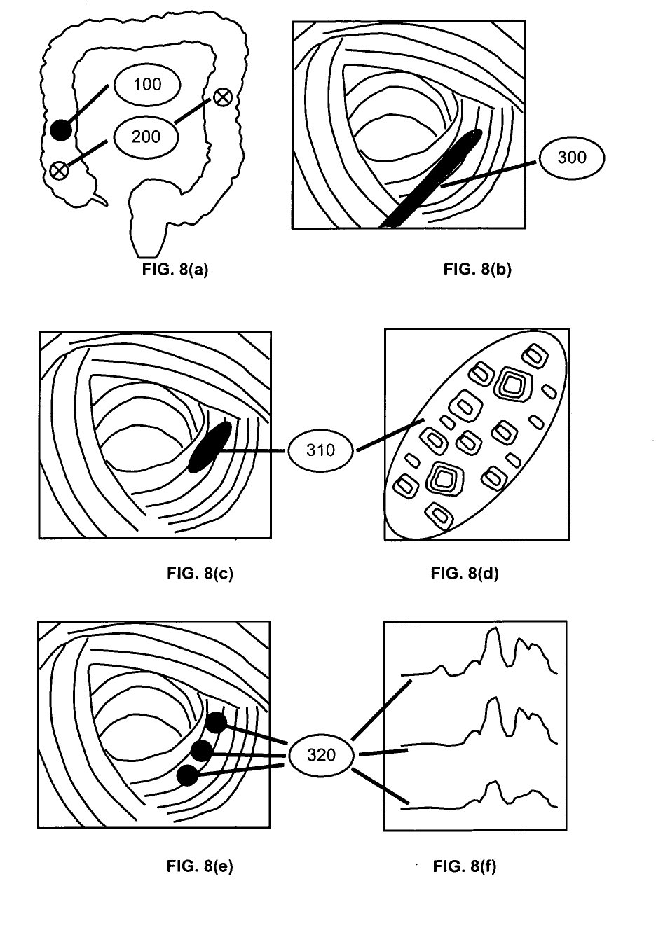

The visualization process allows for spatially registering either by using motion inference algorithms, a tracker system, or a combination thereof (for example as described in D. Sargent, “Endoscope-magnetic tracker calibration via trust region optimization,” Proceedings of SPIE 7625, SPIE Medical Imaging, 76252L1-9, 2010; and D. Sargent, S. Park, I. Spofford, K. Vosburgh, “Image-based endoscope estimation using prior probabilities,” Proc. SPIE 7964, pp. 79641U1-11, 2011, incorporated herin by reference) data obtained from the colonoscopic video scene with data obtained from a co-moving probe onto the digital colon model. The intent is to effectively produce a wide field of view with an ‘x-marks-the-spot’ type symbol indicating the location of the probe. This approach will show where the probe is (or was) during a colonoscopic exam as illustrated in FIG. 8. FIG. 8(a) shows the digital colon model with the position of colonoscope tip (100) and local rendering(s) at locations (200) where the probe is (or was used). FIG. 8(b) shows the traditional colonoscopic video view with the probe tip (300) extended into the video view. As part of the registration process FIG. 8(c) and FIG. 8(e) depict the location of the microscopic (310) and spectroscopic (320) probe data superimposed onto the navigable digital colon model. FIG. 8(d) and FIG. 8(f), respectively, display the magnified view of the imaging data (310) such as acquired from confocal microscopy or optical coherence tomography) and the spectroscopic data (320) such as acquired from infrared spectroscopy.

### Feature Alert System

A feature alert system is preferably used during a clinical exam, but it can also be used on pre-recorded exam data. The alert system preferably automatically interprets each frame in the streaming colonoscopic video data and classifies and annotates the findings. The alert system immediately notifies the physician of any suspicious or anomalous tissue visible in the video data screen while he or she is navigating through the colon. The physician can then temporarily stop the navigation (screening process) and invest more time to fully analyze the tissue in question.

The feature alert system preferably provides an alert list based on the features employed by the video interpretation system, such as the minimal standard terminology for endoscopy and other non-diagnostic features, including but not limited to, frame degradation factors (such as obstructions, blur, glare, and illumination) and objects in the colonoscopic video scene (such as blood, stool, water, and tools). In the preferred embodiment of the feature alert system, the physician can choose to use the entire alert list or a subset by defining and modifying the features of the alert list. When there are matches between the alert list and the video stream, the corresponding alerts or notifications are generated for the physician's attention.

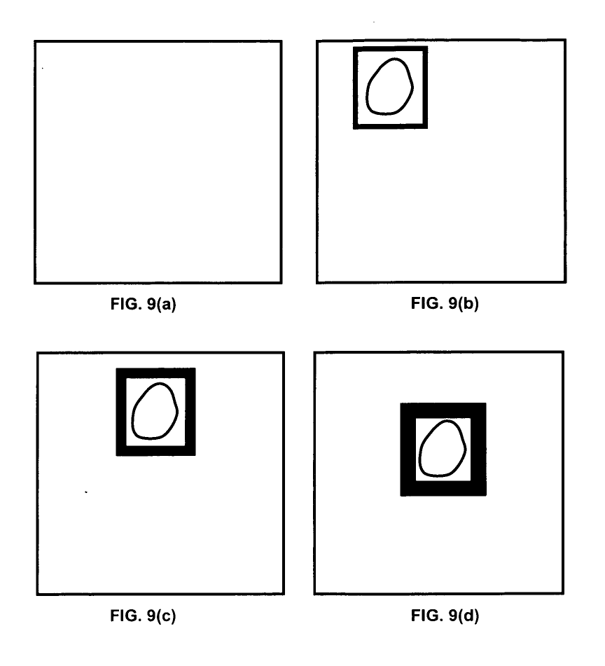

The alerts are preferably defined with different levels representing the severity of the feature. This can be accomplished by utilizing boundaries of different shapes, sizes and colors. For example, as illustrated in FIG. 9 for the alert of a polyp in a colonoscopic video sequence, no alert means no detection (FIG. 9(a)), a black box can indicate the first detection of the feature (see FIG. 9(b)), and increasing line thicknesses of the box can indicate progressively higher probability of detection (see FIG. 9(c) and FIG. 9(d), respectively). Of course, other shapes, size and color schemes for alerts can also be used.

### Detection and Tracking

As a specialized feature of the alert system, detection and tracking for the diagnostically important features of polyps and diverticula can also be applied to the exam video data during or after a colonoscopic exam.

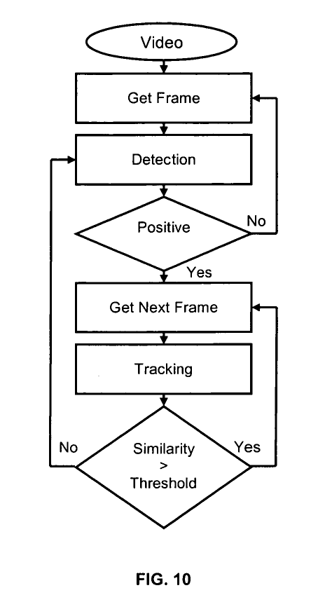

One preferred embodiment of this specialized detection and tracking is to use the classification output of the SSEHMM system. Another preferred embodiment is the application of an unsupervised detection and tracking approach as illustrated in FIG. 10. Detection is enabled to first detect the suspicious tissue. Detection can be either polyps or diverticula, based on the physician's preference. Once a polyp or diverticulum is detected in a video frame, tracking is enabled to track the polyp or diverticulum in subsequent video frames. The quality of tracking is measured by a similarity score ranging from 0 to 1. A higher similarity score indicates a higher probability of tracking the target. The tracking stops when the similarity score is lower than a user-defined threshold, which indicates the polyp or diverticulum is likely no longer in the current frame. When this situation happens, the process starts over with a new detection. To the best of the inventor's knowledge and belief, this is the first report that combines unsupervised detection and tracking of colonic polyps and diverticula in colonoscopic videos.

### Polyp Detection

Polyp detection preferably consists of three major steps applied in sequential order: pre-processing, watershed or other morphological segmentation, and region refinement.

Preprocessing starts with selecting the red channel of a video frame for further analysis to minimize the fine texture from the blood vessels. Next, a Gaussian smoothing function is applied to the red channel image to reduce the noise. Then an adaptive histogram equalization technique (S. M. Pizer, E. P. Ambrun, J. D. Austin, R. Cromartie, A. Geselowitz, T. Greer, B. H. Romeny, J. B. Zimmerman, and K. Zuiderveld, “Adaptive Histogram Equalization and Its Variations,” Computer Vision, Graphics, and Image Processing 39, pp. 355-368, 1987, incorporated herein by reference) is utilized to enhance the background and the local contrast. Since non-uniform lighting conditions are commonly encountered in endoscopic videos, background enhancement is helpful to improve the robustness of polyp detection.

Segmentation preferably utilizes watershed segmentation originally applied to magnetic resonance imagery and digital elevation models (L. Vincent and P. Soille, “Watersheds in Digital Spaces: An efficient algorithm based on immersion simulations,” IEEE Transactions on Pattern Analysis and Machine Intelligence 13, pp. 583-598, 1991; and V. Grau, A. Mewes, M. Alcaniz, R. Kikinis, and S. K. Warfield, “Improved watershed transform for medical image segmentation using prior information,” IEEE Transactions on Medical Imaging 23, pp. 447-458, 2004, incorporated herein by reference).

Region refinement preferably starts by calculating region properties based on their area, average intensity, average color value, solidity, and eccentricity. Regions that satisfy a list of pre-modeled criteria proceed with a shape and texture identification. To refine the polyp candidate regions, two ellipse fitting methods are preferably employed. One method fits an ellipse using the region boundary from the watershed segmentation. The other method fits an ellipse to edges from the general colon structure, which coincide with the region boundary. First, region fitting is performed for the corresponding polyp candidate region. Second, if the fitting error of region fitting is bigger than a pre-defined threshold, the salience fitting is applied.

### Diverticulum Detection

Since diverticulum appears as a dark hole in the video image, the same aproach and processing steps utilized for polyp detection can be applied to the complement of the image to detect diverticulum.

### Tracking

Tracking can be defined as the problem of estimating the trajectory of an object in the image plane as it moves around a scene. In other words, a tracker assigns consistent labels to the tracked object in different frames of a video. The tracking implementation preferably applies a weighted histogram method computed from a circular region to represent the object (D. Comaniciu, V. Ramesh, and P. Meer, “Kernel-Based Object Tracking,” IEEE Transactions on Pattern Analysis and Machine Intelligence, 25, pp. 564-577, 2003, incorporated herein by reference). Another possible approach would be to use template matching, which is a brute force method of searching an image for a region similar to an object template defined in the previous frame. An advantage of the weighted histogram method over template matching is the elimination of the brute force search; instead, the translation of the object path is computed in a small number of iterations.

In the preferred embodiment of the present invention, a target is represented by a rectangle region in a video frame. An isotropic kernel, with a convex and monotonically decreasing kernel profile k(x), with x representing the pixels in the video frame, assigns smaller weights to pixels farther from the center. Using these weights increases the robustness of tracking because the peripheral pixels in a video frame are the least reliable, often being affected by occlusions, deformation, or interference from the background. Meanwhile, the background information is important for two reasons. First, if some of the target features of the polyp or diverticulum are also present in the background, their relevance for localization of the target is diminished. Second, in colonoscopy video data, it is difficult to delineate the target (either polyp or diverticulum) as its model may contain background features as well. Therefore, a background-weighted histogram approach is applied to derive a simple representation of the background features to distinguish them from the representations of the target model and target candidates.

Let ô={ôᵤ}_{u=1K m} with

$${\sum }_{u = 1}^{m} {\hat{o}}_{u} = 1$$

the cuscrete representation of an m-bin histograms of the background in the feature space and ô* be its smallest nonzero entry. The weights are calculated as

$${ν}_{u} = min ( \frac{{\hat{o}}^{*}}{{\hat{o}}_{u}} , 1 ) , \qquad ( 31 )$$

where u=lΛ m.

Furthermore, let {xᵢ*}_{i=1Λ n} be the normalized image pixels located in the target model and k(x)=(∥x∥²) is the selected kernel. The function b: R²→{1Λ m} associates the pixel at location xᵢ* to the index b(xᵢ*) of its bin in the discrete feature space. The target model is then defined as

$${\hat{q}}_{u} = C {ν}_{u} {\sum }_{i = 1}^{n} k ( \vert \vert {x}_{i}^{*} {\vert \vert}^{2} ) δ [ b ( {x}_{i}^{*} ) - u ] \qquad ( 32 )$$

where δ is the Kronecker delta function

$$δ [ k ] = \{ \begin{matrix}1 , k = 0 \\ 0 , k \neq 0\end{matrix} \qquad ( 33 )$$

and the normalization constant C is expressed as

$$C = \frac{1}{{\sum }_{i = 1}^{n} k ( \vert \vert {x}_{i}^{*} {\vert \vert}^{2} ) {\sum }_{u = 1}^{m} {ν}_{u} δ [ b ( {x}_{i}^{*} ) - u ]} \qquad ( 34 )$$

Additionally, let {xᵢ}_{i=1Λ n}_{ₕ} be the normalized pixel locations of the target candidate, centered at y in the current frame. The normalization is inherited from the frame containing the target model. Using the same kernel profile k(x) with bandwidth h, the probability of the feature u=1Λ m in the target candidate is given by

$$\begin{matrix}{\hat{p}}_{u} ( y ) = {C}_{h} {ν}_{u} {\sum }_{i = 1}^{{n}_{h}} k ( \vert \vert \frac{y - {x}_{i}}{h} {\vert \vert}^{2} ) δ [ b ( {x}_{i} ) - u ] \\ where & ( 35 ) \\ {C}_{h} = \frac{1}{{\sum }_{i = 1}^{{n}_{h}} k ( \vert \vert \frac{y - {x}_{i}}{h} {\vert \vert}^{2} ) {\sum }_{u = 1}^{m} {ν}_{u} δ [ b ( {x}_{i} ) - u ]} & ( 36 )\end{matrix}$$

is the normalization constant that can be pre-calculated for a given kernel and different values of bandwidth h. The bandwidth parameter h defines the scale of the target candidate, i.e. the number of pixels considered in the subsequent localization process.

The target localization procedure starts from the position of the target in the previous frame (the model) and searches in the neighborhood. Finding the location corresponding to the target in the current frame is equivalent to maximizing the so-called Bhattacharyya coefficient, which is a measure commonly used in statistics to determine the amount of overlap between two statistical samples (A. Bhattacharyya, “On a measure of divergence between two statistical populations defined by their probability distributions,” Bulletin of the Calcutta Mathematical Society 35, pp. 99-109, 1943. incorporated herein by reference). Therefore the target localization procedure can be formulated as an optimization procedure using a mean shift vector (D. Comaniciu and P. Meer, “Mean shift: a robust approach toward feature space analysis,” IEEE Transactions on Pattern Analysis and Machine Intelligence 24, pp. 603-619, 2002, incorporated herein by reference). At each iteration, the mean shift vector is computed such that the histogram similarity is increased. This process is repeated until convergence is achieved.

Although any color space, including the Red, Green, Blue (RGB) color space most colonoscopic videos are recorded in, can be used in the tracking, the preferred color space is CIE-Lab color space due to its perceptual uniformity. To emphasize the importance of gradient information, a weighted histogram is computed upon the combination of the gradients of the region and the L and a channels of the CIE-Lab color space.

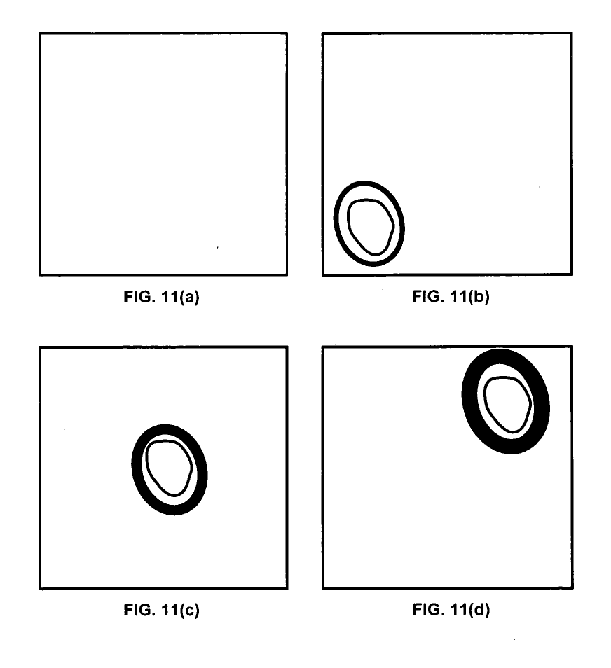

The detection and tracking of polyps or diverticula are displayed in FIG. 11. Similar to feature alerts as described in a previous section, the detection and tracking are preferably represented by a combination of different shapes, sizes and colors. For example, no alert means no detection as illustrated in FIG. 11(a)). The detection of the polyp or diverticulum can be indicated with a black ellipse as shown in FIG. 11(b)). The tracking phase can be indicated with increasing line thicknesses of the ellipse as illustrated in FIG. 11(c) and FIG. 11(d). Of course, other shapes, size and color schemes for alerts can also be used.

### Video Filtering

The purpose of video filtering (the term “filtering” is used here, instead of segmentation, to avoid confusion with watershed or other morphological segmentation and colon segments) as part of a video visualization and management system for colonoscopy is to automatically filter the exam video into clinically relevant and irrelevant video sections, for the purpose(s) of preferentially displaying and/or storing only the relevant portion of the video. For display purposes, minimizing the length of video reduces the physician's time commitment (i.e. maximizes the physician's efficiency) when performing a longitudinal exam comparison or any other review of endoscopic video. Similarly, the elimination of irrelevant section(s) of exam video minimizes the long-term storage requirements, which leads to significant cost savings in medical IT infrastructure.

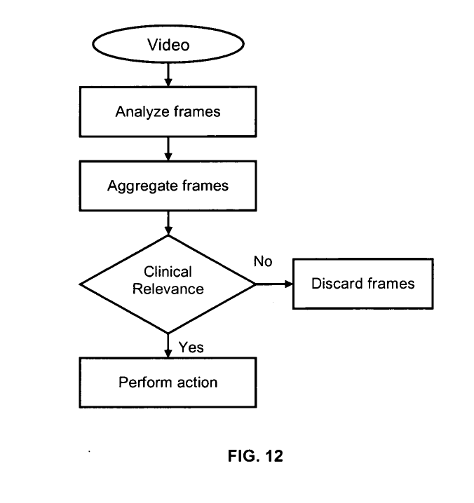

In the preferred embodiment of the visualization and management system, the video filtering is preferably performed using content-based filtering on video data either in real-time during an examination or on pre-recorded examinations, according to the following list of steps as illustrated in FIG. 12:

1. Analyze each video frame or a subset of video frames from the video data to estimate one or more measures of the content of the video frame(s);

2. Aggregate frames into video sections of similar content measure; and

3. Perform one or more actions on the video wherein for each action, the clinical relevance of the content is scored according to a metric for that action, and the action is performed only for those video sections that exceed a threshold for the clinical relevance metric.

There are several possible embodiments of this general process, depending on the approaches selected for analyzing the video and individual video frames: (1) the metrics chosen to quantify the content defined by the approaches, (2) the methods selected for accumulating multiple sequential frames into a continuous video section and (3) the metrics chosen to define the clinical relevance of a particular video section. While the best modes for implementing the process are disclosed by way of example below, such disclosures are not meant to be exclusive of all other possible embodiments of the video filtering method.

### 1. Video Frame Analysis

For frame analysis, one preferred embodiment is to utilize the output from the SSEHMM-based video interpretation system. This system will automatically interpret any video data, output annotations and classifications according to the minimal standard terminology for endoscopy and other features, including but not limited to, frame degradation factors such as obstructions, blur, glare, and illumination, and objects in the colonoscopic video scene such as blood, stool, water, and tools.

Another possible embodiment is to execute several automated image processing algorithms on the input video frames, similar to the approach described in a co-pending, commonly assigned U.S. patent application Ser. No. 11/895,150 for “Computer aided diagnosis using video from endoscopes”, filed Aug. 21, 2006; and EP patent no. 2054852 B1 “Computer aided diagnosis using video from endoscopes,” incorporated herein by reference. For this approach, all of the algorithms, or any subset thereof, can be executed in parallel within the frame analysis module. As opposed to the SSEHMM system that interprets and detects all features at the same time, each algorithm in this approach measures a particular type of content only.

For any embodiment, the content measure for a particular feature reflects how much of the feature is present in the analyzed frames. This content measure can be a simple binary score of either “true” or “false”. Alternatively, the content score may incorporate the uncertainties inherent in any measurement by producing a probability value (0% to 100%) describing to what extent one or more features may be visible in the frame. In addition, the content score can take into account the clinical relevance of the feature, assigning a relevance value (0% to 100%) as to whether the features are important to the physician.

Just as the SSEHMM based video interpretation system incorporates feedback from physicians, the video frame analysis can benefit from physician input to infer the clinical relevance of particular video frames. This input may come in the form of manual input to mark features of frames within the video of anatomical or diagnostic importance. The exact form of input, for example graphical, verbal, or otherwise, is irrelevant to the content-based frame analysis. By way of example, several forms of manual physician input are useful: anatomical landmarks, distal end of organ under examination, and lesions and abnormalities.

The content measure for the physician input is entirely similar and straightforward to the feature content score: the content measure is a binary score that indicates the presence or absence of the particular physician input. In the case of anatomical landmarks in colonoscopy, the ileocecal valve (24), or alternatively the appendiceal orifice (25), as illustrated in FIG. 6, indicates the distal end of the colon. The clinical relevance of this input is that it indicates the end of the insertion phase and beginning of the withdrawal phase of the colonoscopy. In the case of lesions and abnormalities, the primary difference compared to the feature content measure is that the physician has performed the analysis and the input is taken to be correct, i.e. 100% probability of detection, so the content score is binary: presence or absence.

### 2. Video Frame Aggregation

Once each individual frame is scored for each particular type of content under analysis, it is necessary to aggregate the video frames into discrete video sections of similar content measure. This step of frame aggregation for the purpose of video sectioning is performed independently for each specific type of content. It is acceptable and common that multiple overlapping video sections will be created, each based on a different type of content. One possible preferred embodiment for this frame aggregation algorithm is to perform the following steps for each specific content type X determined by frame analysis:

A. Mark the first frame of video as the first frame in the initial video section and categorize this initial section as “containing content type X” or “not containing content type X” using the result of the first frame analysis.

B. Check the subsequent frame analysis result against the video section category. If they are the same, consider the frame to be part of the current section. If they are different, create a new video section starting with the new frame by marking the new frame as the end of the current section and the start of a new section, and categorize the new section according to the new frame's analysis result.

C. Continue to apply step 2 to subsequent video frames until the end of the video is reached.

D. Upon completion, ignore the start and end marks for video sections that are categorized as “not containing content type X”. The remaining start and end marks define all video sections containing content type X.

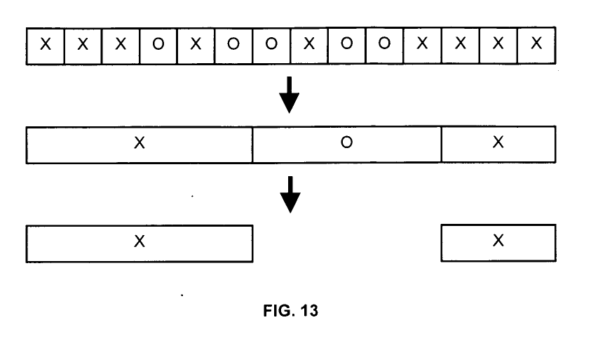

Another possible preferred embodiment is to extend the first approach to require that N, rather than 1, consecutive frames of the opposite category must occur before marking the end of the current video section and the start of a new one, where N is a configurable positive integer. If N or more consecutive frames of the opposite category do occur, the new video section starts on the first frame of the opposite category. FIG. 13 graphically depicts this embodiment for N=2 with X illustrating video frames “containing content type X” and O illustrating vide frames “not containing content type X”.

Yet another possible preferred embodiment is to extend the previous approach so that the threshold of consecutive frames to go from a video section “containing content type X” to a section “not containing content type X” is N, and the threshold to go from a video section “not containing content type X” to a section “containing content type X” is M, where M and N are possibly different positive integers.

Note that all of the above preferred embodiments assume that the frame analysis outputs are binary scores, either “containing content type X” or “not containing content type X”. For the more general case of continuous content scores from the frame analysis, the possible embodiments may preferably include, but are not limited to, the determination of video section content according to a pre-configured threshold. For instance, if the score for content type X of a video frame is at or above a threshold T, then the video section containing that frame is categorized as “containing content type X”. Otherwise, the score is below the threshold T and the section is categorized as “not containing content type X”.

Furthermore, note that all of the above embodiments result in video sections that do not overlap for a specific content type X, though it is likely that these sections will overlap with those of a different content type Y. For content analyses where the result may indicate multiple instance of the content in a single frame, the possible embodiments may include, but are not limited to, the following:

A. Both the frame analysis and the frame aggregation treat all instances of the content as the same content type X. The frame analysis step marks a frame as containing content type X if one or more instances of that content, e.g. one or more polyps, are present in the frame. The frame aggregation method performs as described, thereby resulting in a single video section for multiple overlapping instances of the same content type X in the endoscopic exam video.

B. Both the frame analysis and the frame aggregation treat each instance of the content as a different content type, e.g. X1 and X2. The frame analysis step marks a frame as containing content type X1 for the first instance of that content, e.g. the first polyp, it marks a frame as containing content type X2 for the second instance of that content, and it continues in this fashion until all instances have been marked. The frame aggregation methods performs as described, treating each instance as a different content type, thereby resulting in a single video section for each instance of the overall content type X in the endoscopic exam video. For this case, different section for the overall content type X may overlap.

### 3. Preferential Execution of Actions on Clinically Relevant Video Sections

The final step in this filtering process is to perform a specific action on the endoscopic exam video. The action is executed preferentially on only those video sections that are deemed to have “clinical relevance”. “Clinical relevance” is defined at the time of the action execution, and it consists of an arbitrary logical combination of content types. Since the clinical relevance is determined every time an action is executed, it may be configured or modified every time an action is executed. An alternative embodiment is to statically define the clinical relevance for an action or a subset of actions, so that the same metric is applied every time the action or actions are executed.

Actions include, but are not limited to, video storage on a computer medium (such as a hard disk, thumb drive, picture archiving and communication system (PACS), or otherwise) and video playback for review by the physician.

One possible embodiment comprises: a computer program statically defines the clinical relevance metric to be applied for storing a colonoscopic exam video to a PACS server. The metric is defined as excluding all content except the withdrawal phase of the colonoscopic examination. The presence or absence of this content is determined by a frame analysis module that checks for a physician's input that marks the frame with a view of the ileocecal valve, i.e. the distal end of the organ under examination. The analysis module marks all frames before this frame is received as “insertion phase” and marks the marked frame and all subsequent frames as “withdrawal phase”. Therefore, the frame aggregation module will create a single video section for the withdrawal phase that corresponds to the latter portion of the video after the ileocecal valve. Whenever an exam video is stored, only the latter portion of the video will be saved to PACS.

Another possible embodiment comprises: a physician decides, through the aid of a computer program, to play only the portions of a past colonoscopic examination video that contain polyps. The computer program enables the physician to configure playback of polyp video sections only, whereas the previous playback of the same or different video may have been configured to play all in-focus video sections. A polyp detection module (based on preferably either the SSEHMM interpretation system or the unsupervised detection and tracking approach previously described) perform the frame analysis to mark any frame containing one or more polyps, and the frame aggregation module creates multiple video sections if the examination reveals one or more polyps at multiple locations in the colon. Playback will only show sections containing one or more polyps and will skip all other sections.

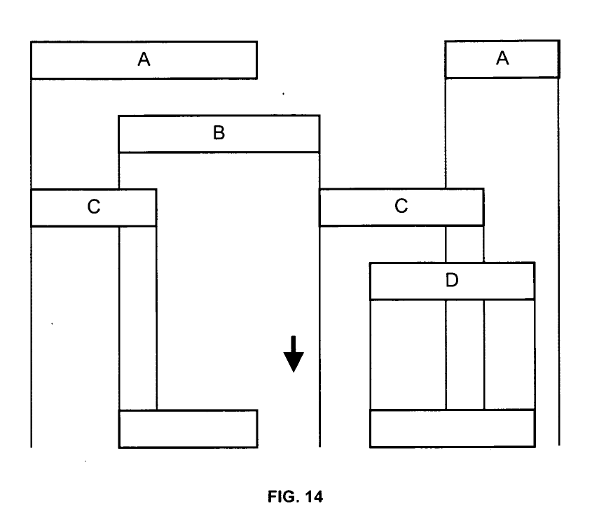

FIG. 14 graphically depicts a more general embodiment, where there are 4 different content-based frame analyses and the physician desires to perform an action on all sections that contain content types (A and B) or D. In particular, this embodiment demonstrates how the final step may create new video sections based on a logical combination of the content-based video sections.

### Video Spatial Synchronization

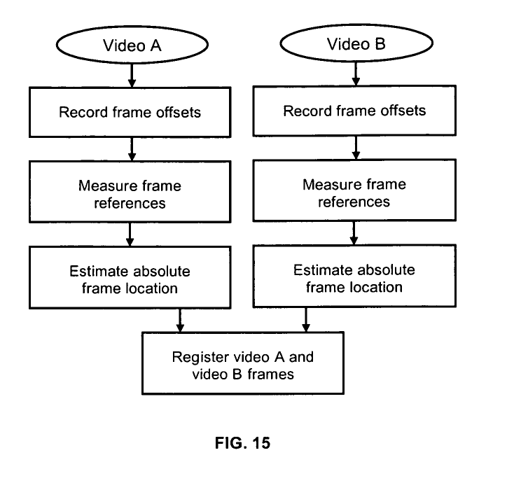

The purpose of video spatial synchronization is to synchronize the spatial location in multiple videos that all contain footage of the same scene. In this context, “synchronize” means to display the same spatial location of the object under investigation, such as the colon, simultaneously within each video, rather than the usual definition of temporal alignment. The process involves four independent steps as illustrated in FIG. 15 for two different videos A and B. The first three steps are performed independently on each video as it is originally captured:

(1) record the frame (or time) offsets within the video of a series of absolute (i.e. global) spatial reference measurements;

(2) measure and/or estimate the (local) spatial reference of each frame relative to the previous frame;

(3) optimally estimate the absolute (i.e. global) spatial location of every video frame from the measurements obtained in steps 1 and 2.

The final step involves pairs of videos:

(4) register the current frame in video A to the frame in video B that most closely matches.

In this novel process, only step 1, 2, or 4 is required, and the remaining steps are optional and expected to improve the accuracy of synchronization.

For example, a possible implementation of this process is during longitudinal exam review of two colonoscopic videos: while viewing a specific location within the video of one (possibly ongoing) exam, the physician can quickly review the video of the same location from a different exam. Using this embodiment, the details of each step in the process are illustrated as follows:

Step 1 serves to “tag” a number of frames with absolute spatial location information. Though it is not a restriction of this process, it is often considered that these tags are quite accurate, but coarsely spaced both spatially and temporally. Anatomical landmarks during colonoscopy are an excellent example of this process step: as the live video is displayed during capture, relevant “landmarks” within the colon, such as the anus (20), splenic flexure (22), hepatic flexure (23), ileocecal valve (24), and/or appendiceal orifice (25) as illustrated in FIG. 6 can be marked. The means of marking these landmarks, e.g. automatically, graphical, verbal, or otherwise, is not relevant to the process. These landmarks serve to “anchor” the colon video at several points, but do not provide any further location information between landmarks.

Another example of this first process step is a tracker system that measures the absolute location of the endoscope tip. In this case, the spatial location measurement may have a varying uncertainty associated with it, and the measurements may be finely spaced, e.g. on every frame.

Step 2 provides a relative measurement between subsequent frames of video. Thus, a dead-reckoning approach can be utilized that accumulates these measurements to estimate the absolute spatial location of every frame of video. Dead reckoning is the process of estimating the current position based upon a previously determined position, or fix, and advancing that position based upon known or estimated speeds over elapsed time, and course. However, the errors in the resulting absolute measurements are subject to increase without bound in a random-walk fashion as the number of frames increases. Video-based motion inference techniques fall under this process step—the frame-to-frame registration of features, textures, etc. effectively produces a relative spatial location measurement and associated uncertainty.

Step 3 integrates the measurements of steps 1 and 2 in a sensor fusion process (provided that both steps 1 and 2 are included in the given embodiment of this novel process). Assuming that both sets of measurements include associated uncertainty estimates, optimal estimation techniques can be utilized to provide predictions for the absolute spatial locations of every video frame, where these predictions are more accurate than either set of measurements alone. In this sense, “optimal” is used rather loosely—this process step encompasses any method that intelligently combines the two input measurement sets to form a superior (i.e. “optimal” according to some metric) set of estimates. In continuation of the examples illustrated in steps 1 and 2, video-based motion inference and landmarks can be combined optimally along the length of the lumen. In essence, the landmark locations provide “anchor points” to reset the dead-reckoning error that accumulates when using relative frame-to-frame measurements.

Step 4 takes a different approach to the spatial synchronization problem than steps 1-3. This process step directly compares a frame of video to one or more frames in one of the other videos to be synchronized. For instance, within a set of endoscopic exam videos, a variety of feature-matching techniques could be utilized to find which frame in video B matches the current frame from video A. This process step makes the implicit assumption that corresponding frames in two different videos that provide the best “match” represent identical spatial locations. This approach works similarly for multiple videos by simply performing pairwise video synchronization between the different videos, first video A to video B, then video B to video C, followed by video C to video D, until all videos have been synchronized to the current “master” frame from the “master” video (in this example video A). It is important to note that the search space for finding these cross-references can be bounded significantly by incorporating the results from steps 1-3, thereby improving the accuracy and computational efficiency of this process step. Of course, this improvement is not a required part of the process, and step 4 can stand alone as one possible embodiment.

### Field of View Visualization Scoring

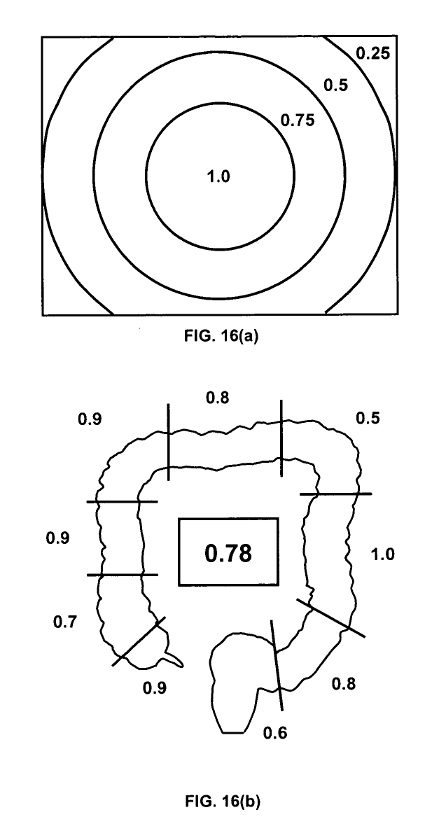

Endoscopic video is captured at a wide field of view, 140 or higher degrees. Analogous with the video filtering content score, as described in a previous section, an automated weighting system is disclosed which preferably considers the center field of view (60 degrees) of highest value, assigning it a score. Every 20 degree increase in field of view, envisioned as concentric rings around the center, are progressively decreasing in weighting score. FIG. 16(a) graphically depicts this scoring scheme with the 60° center field of view being assigned a score of 1.0 and each twenty degree increase in field of view decreases the score by 0.25. Since the endoscope tip orientation is controllable, this could enable an automatic feedback loop to the physician to ensure they “paint” the entire colonoscopic video scene to maximize their visualization score. The output could be displayed with a color coding or grayscale value.

One possible embodiment is as follows: The first step in the field-of view visualization scoring is to utilize the previously described digital colon, or any other realization of a generic colon. Then, as the colonoscope traverses the colon, each video frame will be registered within the digital colon model. Since each pixel in an image frame can be assigned a “score” based on its angle away from the image center, the corresponding mapped location in the digital colon model will receive the same score. The resulting digital colon model contains high scores where that area of the colon was seen near the center of a frame of video, whereas extremely low scores indicate locations in the colon model that were seen only at an oblique angle (or never seen at all) in the video. A resulting score for the entire exam are then the average of the scores for each video frame. This is illustrated in FIG. 16(b) where different sections of the colon have been assigned scores between zero and 1. Also shown in FIG. 16(b) is the exam score, which is the average of the score for the different video sections ([0.6+0.8+1.0+0.5+0.8+0.9+0.9+0.7+0.9]/9=0.78).Of course, other schemes, such as color codes or grayscale value can be used for the scoring.

### INDUSTRIAL APPLICABILITY

This invention provides the means to interpret, visualize, assess the quality, and manage colonoscopic exams, videos, images and patient data. The methods described may also be suitable for other medical endoscopic applications and other non-medical video and imaging systems that are designed to interpret, visualize, and manage video and imagery. For example, the methods described may be used in automatic guidance of vehicles, examination of pipelines, or other fields where objects and features in video data need to be recognized and classified.

What is claimed is:

1. A process for detecting colon cancer by identifying clinical features in a colon, comprising:
obtaining multiple colonoscopy video frames containing colonoscopic features;
applying a probabilistic analysis to intra-frame relationships between colonoscopic features in spatially neighboring portions of said video frames, and to inter-frame relationships between colonoscopic features in temporally neighboring portions of said video frames; and
classifying and annotating as clinical features any of said colonoscopic features that satisfy said probabilistic analysis as clinical features.

2. A process according to claim 1, wherein said probabilistic analysis is selected from the group consisting of Hidden Markov Model analysis and a conditional random field classifier.

3. A process according to claim 1, further comprising:
training a computer to perform said probabilistic analysis by semi supervised learning from labeled and unlabeled examples of clinical features in video frames containing colonoscopic features.

4. A process according to claim 3, wherein said training step further comprises physician feedback.

5. A process according to claim 1, further comprising applying a forward-backward algorithm and model parameter estimation.

6. A process according to claim 1, further comprising additionally applying augmenting probabilistic analysis to at least one additional dimension of relationships between said colonoscopic features selected from the group consisting of frame quality, anatomical structures, and imaging multimodality.

7. A process according to claim 6, wherein said additional applying step is applied in a hierarchical manner first to video quality, then to anatomical structures, then to multimodalities.

8. A process for detecting colon cancer by identifying clinical features in a colon, comprising:
training a computer to perform probabilistic analysis by semi supervised learning from labeled and unlabeled examples of clinical features in video frames containing colonoscopic features;
obtaining multiple colonoscopy video frames containing colonoscopic features;
excluding any uninformative video frames;
applying a probabilistic analysis selected from the group consisting of Hidden Markov Model analysis and conditional random field classifier to five dimensions of relationships between colonoscopic features in temporally or spatially neighboring portions of said video frames;
wherein said five dimensions of relationships consist of inter-frame relationships, intra-frame relationships, frame quality, anatomical structures, and imaging modalities; and
classifying and annotating any of said colonoscopic features in said video frames that satisfy said probabilistic analysis as clinical features.

9. A process according to claim 8, further comprising pre-processing said video frames before said applying step, wherein said pre-processing step is selected from the group consisting of detecting glare regions, detecting edges, detecting potential tissue boundaries, correcting for optical distortion, de-interlacing, noise reduction, contrast enhancement, super resolution and video stabilization.

10. A process according to claim 8, further comprising providing progressively decreasing weighting scores as the field of view of said video frames increases.

11. A process according to claim 8, further comprising filtering said video frames into clinically relevant and clinically irrelevant sections and displaying or storing only frames that exceed a threshold for clinical relevance, wherein said filtering step is performed by:
analyzing said video frames to estimate at least one measure of content of each of said video frames;
aggregating frames into sections of similar content measure; and
performing at least one action on frames that exceed a threshold for said clinical relevance metric, wherein clinical relevance of said content of each frame is scored according to a metric for that action.

12. A process according to claim 8, further comprising providing a generic digital colon model for visual navigation through colon videos.

13. A process according to claim 12, wherein said clinical features are registered within said generic digital colon model.

14. A process for detecting and tracking polyps and diverticula in colonoscopic video, comprising:
pre-processing said video to enhance contrast;
segmenting said video to identify regions of interest;
refining said regions of interest by similarity scores in subsequent video frames to determine a final region of interest;
estimating a trajectory of said final region of interest between video frames in said video.

15. A process for video spatial synchronization of at least two colonoscopic videos, comprising:
tagging spatially and temporally coarsely spaced video frames with spatial location information in each video;
estimating positions of frames subsequent to said tagged video frames in each video; and
registering frames in said videos having most closely matching features.

16. A device for detecting colon cancer by identifying clinical features in a colon, comprising:
obtaining means for obtaining multiple colonoscopy video frames containing colonoscopic features;
excluding means for excluding any uninformative video frames;
applying means for applying a probabilistic analysis selected from the group consisting of Hidden Markov Model analysis and conditional random field classifier to five dimensions of relationships between colonoscopic features in temporally or spatially neighboring portions of said video frames;
wherein said five dimensions of relationships consist of inter-frame relationships, intra-frame relationships, frame quality, anatomical structures, and imaging multimodalities;
classifying and annotating means for classifying and annotating any of said colonoscopic features in said video frames that satisfy said probabilistic analysis as clinical features;
filtering means for creating sections of said video containing relevant clinical features;
wherein said probabilistic analysis has been trained by semi supervised learning from labeled and unlabeled examples of clinical features in video containing colonoscopic features;
storage means for capturing, storing, searching and retrieving clinically relevant video frames;
feature alert means for automatically interpreting, classifying and annotating said video frames; and
field of view scoring means for scoring field of view of said video frames.
